SEQUENCE LISTING (28 sequences)

SEQ ID NO: 1 - DNA, 20 bp, synthetic construct
  source 1..20: mol_type = other DNA
aactttggcattgtggaagg

SEQ ID NO: 2 - DNA, 20 bp, synthetic construct
  source 1..20: mol_type = other DNA
acacattgggggtaggaaca

SEQ ID NO: 3 - DNA, 26 bp, synthetic construct
  source 1..26: mol_type = other DNA
tcatgaagtgtgacgttgacatccgt

SEQ ID NO: 4 - DNA, 26 bp, synthetic construct
  source 1..26: mol_type = other DNA
cctagaagcatttgcggtgcacgatg

SEQ ID NO: 5 - DNA, 22 bp, synthetic construct
  source 1..22: mol_type = other DNA
catgtaaaggagataaggaact

SEQ ID NO: 6 - DNA, 19 bp, synthetic construct
  source 1..19: mol_type = other DNA
atgaatgcgtcagattggg

SEQ ID NO: 7 - DNA, 21 bp, synthetic construct
  source 1..21: mol_type = other DNA
tcctcaaaagaaaccgtgcat

SEQ ID NO: 8 - DNA, 26 bp, synthetic construct
  source 1..26: mol_type = other DNA
agattaatggttgctaaggactggat

SEQ ID NO: 9 - DNA, 20 bp, synthetic construct
  source 1..20: mol_type = other DNA
ctggagctctacagtgatga

SEQ ID NO: 10 - DNA, 22 bp, synthetic construct
  source 1..22: mol_type = other DNA
cagttaccaatcaacacatcac

SEQ ID NO: 11 - DNA, 22 bp, synthetic construct
  source 1..22: mol_type = other DNA
atcttgtcctcaacctcagact

SEQ ID NO: 12 - DNA, 23 bp, synthetic construct
  source 1..23: mol_type = other DNA
ttctttagcctggctttctttca

SEQ ID NO: 13 - DNA, 20 bp, synthetic construct
  source 1..20: mol_type = other DNA
tggccctgatcgactgtaat

SEQ ID NO: 14 - DNA, 20 bp, synthetic construct
  source 1..20: mol_type = other DNA
tggtgcatccaacctaactg

SEQ ID NO: 15 - DNA, 22 bp, synthetic construct
  source 1..22: mol_type = other DNA
tgttagttgccacaccaggacg

SEQ ID NO: 16 - DNA, 22 bp, synthetic construct
  source 1..22: mol_type = other DNA
tggtggcattgtgtcctgctca

SEQ ID NO: 17 - DNA, 22 bp, synthetic construct
  source 1..22: mol_type = other DNA
tgctggctatcttggaggacgt

SEQ ID NO: 18 - DNA, 22 bp, synthetic construct
  source 1..22: mol_type = other DNA
gcggttgtttccacctcttgca

SEQ ID NO: 19 - DNA, 20 bp, synthetic construct
  source 1..20: mol_type = other DNA
tctttaatagtaaaagctga

SEQ ID NO: 20 - DNA, 20 bp, synthetic construct
  source 1..20: mol_type = other DNA
gatgaagacgctgttgaaca

SEQ ID NO: 21 - DNA, 20 bp, synthetic construct
  source 1..20: mol_type = other DNA
acagtttacagactgactac

SEQ ID NO: 22 - DNA, 20 bp, synthetic construct
  source 1..20: mol_type = other DNA
ggacctttacagccttaata

SEQ ID NO: 23 - DNA, 20 bp, synthetic construct
  source 1..20: mol_type = other DNA
ttcagttgctacagtagacg

SEQ ID NO: 24 - DNA, 20 bp, synthetic construct
  source 1..20: mol_type = other DNA
tcatttagcctctgaattcg

SEQ ID NO: 25 - DNA, 23 bp, synthetic construct
  source 1..23: mol_type = other DNA
caccggagttaatataaaaaaca

SEQ ID NO: 26 - DNA, 23 bp, synthetic construct
  source 1..23: mol_type = other DNA
aaactgttttttatattaactcc

SEQ ID NO: 27 - DNA, 24 bp, synthetic construct
  source 1..24: mol_type = other DNA
caccggcagcgcggctatcgtggc

SEQ ID NO: 28 - DNA, 24 bp, synthetic construct
  source 1..24: mol_type = other DNA
aaacgccacgatagccgcgctgcc

(19) United States
(12) Patent Application Publication
(10) Pub. No.: US 2026/0357321 A1
(43) Pub. Date: Nov. 12, 2026

(54) Y CHROMOSOME GENE SIGNATURE AS A TARGET FOR CANCER THERAPY IN COMBINATION WITH IMMUNOTHERAPY

(71) Applicants: CEDARS-SINAI MEDICAL CENTER, Los Angeles, CA (US); OHIO STATE INNOVATION FOUNDATION, Columbus, OH (US)

(72) Inventors: Dan THEODORESCU, Los Angeles, CA (US); Hany ABDEL-HAFIZ, Los Angeles, CA (US); Johanna M. SCHAFER, Tucson, AZ (US); Zihai LI, Powell, OH (US); Jonathan KAYE, Carlsbad, CA (US)

(73) Assignees: CEDARS-SINAI MEDICAL CENTER, Los Angeles, CA (US); OHIO STATE INNOVATION FOUNDATION, Columbus, OH (US)

(21) Appl. No.: 19/483,745

(22) PCT Filed: May 13, 2024
(86) PCT No.: PCT/US2024/029111

§ 371 Date: Nov. 12, 2025

Related U.S. Application Data
(60) Provisional application No. 63/466,556, filed on May 15, 2023.
(60) Provisional application No. 63/570,132, filed on Mar. 26, 2024.

(51) Int. Cl. C12Q 1/68; A61K 38/00; A61P 35/04; C07K 16/28

(52) U.S. Cl. CPC C12Q 1/68; A61K 38/00; A61P 35/04; C07K 16/2827; C12Q 2600/106; C12Q 2600/158; G01N 2800/52; G01N 2800/7028

(57) ABSTRACT

Described herein are methods of selecting treatment for bladder cancer and the treatment of bladder cancer. The treatment of the bladder cancer is based on a Y chromosome gene expression signature in a biological sample obtained from the subject.

### CROSS-REFERENCE TO RELATED APPLICATIONS

This application includes a claim of priority under 35 U.S.C. § 119(e) to U.S. provisional patent application No. 63/466,556, filed May 15, 2023, and U.S. provisional patent application No. 63/570,132, filed Mar. 26, 2024, the entirety of both which are hereby incorporated by reference.

### STATEMENT REGARDING FEDERALLY SPONSORED RESEARCH OR DEVELOPMENT

This invention was made with Government support under Grant Nos. CA143971, CA262069, CA262388 and AI077283, awarded by National Institutes of Health. The Government has certain rights in the invention.

### REFERENCE TO SEQUENCE LISTING

This application contains a Sequence Listing submitted as a computer readable form named “065472-000915WOPT.xml”, having a size in bytes of 25,794 bytes, and created on May 7, 2024. The information contained in this computer readable form is hereby incorporated by reference in its entirety.

### FIELD OF INVENTION

This invention relates to patient selection and the treatment of cancers.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Cancers, including bladder cancer (BLCA) is a major public health problem. It is useful for clinicians to know how aggressive patients' cancer in order to determine the type of the treatment. Thus, there is an unmet need to identify diagnostic biomarkers that can be used in conjunction with existing treatment methods to determine whether the patient has or continue to have cancer, such as bladder cancer, so that the patients in need of treatment can be identified at an early stage.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a method of selecting a cancer treatment for a male subject, comprising: assaying a biological sample obtained from the male subject to determine a Y chromosome gene expression signature; detecting a loss of Y chromosome (LOY) phenotype, and selecting a therapy comprising an immune checkpoint inhibitor, a TOX inhibitor, or a combination thereof, or detecting a Y chromosome high phenotype, and selecting a therapy comprising an immune checkpoint inhibitor and an inhibitor of one or more genes on the Y Chromosome.

In various embodiments, the cancer can be bladder cancer (BLCA), colon adenocarcinoma microsatellite instability (COAD MSI), skin cutaneous melanoma (SKCM), kidney renal clear cell carcinoma (KIRC), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD).

In various embodiments, detecting LOY phenotype can comprise detecting a low level of one or more genes on the Y chromosome, as compared to each gene's reference level.

In various embodiments, the one or more genes can comprise BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the one or more genes can comprise BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the one or more genes can comprise KDM5D, UTY (KDM6C), TBL1Y, ZFY, or a combination thereof. In various embodiments, the one or more genes can comprise UTY, KDM5D, or both. In various embodiments, the one or more genes can be NLGN4Y.

In various embodiments, the biological sample can be a tumor sample.

In various embodiments, the immune checkpoint inhibitor can be an anti-PD-L1 inhibitor or an anti-PD-1 inhibitor. In various embodiments, the immune checkpoint inhibitor is atezolizumab.

In various embodiments, the method can further comprise administering the selected therapy.

Various embodiments of the invention provide for a method of treating cancer in a male subject, comprising: administering a therapy comprising an immune checkpoint inhibitor, a TOX inhibitor, or a combination thereof to the male subject, wherein the male subject has been detected to have a loss of Y chromosome (LOY) phenotype, or administering a therapy comprising immune checkpoint inhibitor and an inhibitor of one or more genes on the Y chromosome to the male subject, wherein the male subject has been detected to have a Y chromosome high phenotype.

In various embodiments, the cancer can be bladder cancer (BLCA), colon adenocarcinoma microsatellite instability (COAD MSI), skin cutaneous melanoma (SKCM), kidney renal clear cell carcinoma (KIRC), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD).

In various embodiments, the inhibitor can be an inhibitor of BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the inhibitor can be an inhibitor of BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the inhibitor can be an inhibitor of KDM5D, UTY (KDM6C), TBL1Y, or ZFY. In various embodiments, the inhibitor can be an inhibitor of UTY, KDM5D, or both. In various embodiments, the inhibitor can be an inhibitor of NLGN4Y.

In various embodiments, the immune checkpoint inhibitor can be an anti-PD-L1 inhibitor or an anti-PD-1 inhibitor. In various embodiments, the immune checkpoint inhibitor can be atezolizumab.

Various embodiments provide for a method of treating cancer in a male subject, comprising: requesting the results of an assay of a biological sample obtained from the male subject to determine a Y chromosome gene expression signature; administering a therapy comprising an immune checkpoint inhibitor, a TOX inhibitor, or a combination thereof to the male subject, wherein the male subject has been detected to have a loss of Y chromosome (LOY) phenotype, or administering a therapy comprising immune checkpoint inhibitor and an inhibitor of one or more genes on the Y chromosome to the male subject, wherein the male subject has been detected to have a Y chromosome high phenotype.

In various embodiments, the cancer can be bladder cancer (BLCA), colon adenocarcinoma microsatellite instability (COAD MSI), skin cutaneous melanoma (SKCM), kidney renal clear cell carcinoma (KIRC), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD).

In various embodiments, the LOY phenotype can be detected by detecting a low level of one or more genes on the Y chromosome, as compared to each gene's reference level.

In various embodiments, the one or more genes can comprise BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the one or more genes can comprise BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the one or more genes can comprise KDM5D, UTY (KDM6C), TBL1Y, ZFY, or a combination thereof. In various embodiments, the one or more genes can comprise UTY, KDM5D, or both. In various embodiments, the one or more genes can be NLGN4Y.

In various embodiments, the biological sample can be a tumor sample.

In various embodiments, the inhibitor can be an inhibitor of BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the inhibitor can be an inhibitor of BPY2, CDY1B, CYorf15A, CYorf15B, DAZ1, DAL2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the inhibitor can be an inhibitor of KDM5D, UTY (KDM6C), TBL1Y, ZFY, or a combination thereof. In various embodiments, the inhibitor can be an inhibitor of UY, KDM5D, or both.

In various embodiments, the immune checkpoint inhibitor can be an anti-PD-L1 inhibitor or an anti-PD-1 inhibitor. In various embodiments, the immune checkpoint inhibitor can be atezolizumab.

Various embodiments provide for a method of determining susceptibility to treatment with an immune checkpoint inhibitor, a TOX inhibitor, or both, in a male subject having cancer, comprising assaying a biological sample obtained from the male subject to determine a Y chromosome gene expression signature; detecting a loss of Y chromosome (LOY) phenotype, wherein the LOY indicates a susceptibility treatment with an immune checkpoint inhibitor, a TOX inhibitor, or both, or detecting a Y chromosome high phenotype, wherein the Y chromosome high phenotype indicates a low likelihood of susceptibility treatment with an immune checkpoint inhibitor, a TOX inhibitor, or both, or indicates a benefit from a therapy comprising an immune checkpoint inhibitor and an inhibitor of the one or more genes on the Y chromosome.

In various embodiments, the cancer is bladder cancer (BLCA), colon adenocarcinoma microsatellite instability (COAD MSI), skin cutaneous melanoma (SKCM), kidney renal clear cell carcinoma (KIRC), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD).

In various embodiments, detecting the LOY phenotype comprises detecting a low level of one or more genes on the Y chromosome, as compared to each gene's reference level.

In various embodiments, the one or more genes comprise BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the one or more genes comprise BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the one or more genes comprise KDM5D, UTY (KDM6C), TBL1Y, ZFY, or a combination thereof.

In various embodiments, the one or more genes comprise UTY, KDM5D, or both.

In various embodiments, the one or more genes is NLGN4Y.

In various embodiments, the biological sample is a tumor sample.

In various embodiments, the immune checkpoint inhibitor is an anti-PD-L1 inhibitor or an anti-PD-1 inhibitor.

In various embodiments, the immune checkpoint inhibitor is atezolizumab.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (panels a-c) shows that LOY is associated with a worse prognosis for men with MIBC. a, Heatmap of clinical parameters and metadata for 300 male patients with MIBC from TCGA data. b-c, Plot of Y chromosome gene expression (b) and Kaplan-Meier survival curve (c) associated with Y_{high} (n=182) and Y_{low} (n=118) samples identified in a. Differences in gene expression and survival were based on Wilcoxon rank-sum test and Logrank statistics, respectively. Boxplots represent the mean with first and third quartile data. Minimum and maximum datapoints are included.

FIG. 2 (panels a-h) show that LOY and deletion of Y chromosome-encoded genes Kdm5d and Uty promotes bladder tumor growth in the immune competent host. a, Y+ and Y− MB49 in vitro cell proliferation (MTT cell viability) over a 6-day time course. Data are mean±s.e.m. n=3 biological replicates. b-c, Tumor volume of Y+ and Y− MB49 grown subcutaneously in WT C57BL/6 (n=10 mice per group) or Rag2^{-/-}1l2rg^{-/-} male mice (n=10 mice per group). d, Relative mRNA expression (qRT-PCR) of Uty, Kdm5d, Eifs3y, and Ddx3y in CRISPR-generated Y-Scr and Y-KO MB49 lines. e, Tumor volume of CRISPR-Y-Scr and CRISPR-Y-KO MB49 grown subcutaneously in WT C57BL/6 male mice (n=10 and 8 mice per group, respectively). f, Volcano plot of statistically significant (Benjamini-Hochberg method, P <0.05) DEGs from Y+ (blue) and Y− (red) MB49 cells that have a |>1 log 2 fold-change in expression. g, Principal component analysis (PCA) of DEGs described in f. h, Tumor volume of Y+, Uty KO (knockout), and Kdm5d KO lines injected subcutaneously into WT (n=10 mice per group) or Rag2^{-/-}Il2rg^{-/-} male mice (n=10 mice per group). Y−, Uty OE (overexpression), and Kdm5d OE cell lines were implanted into WT mice (n=10 mice per group) or Rag2^{-/-}Il2rg^{-/-} male mice (n=8, 6 and 6 mice per group, respectively). All mouse experiments are representative of two independent experiments and statistical significance was determined by repeated measures two-way AVOVA. Statistical comparisons were only made with the Y+ and Y− controls in h. Data are mean±s.e.m.

FIG. 3 (panels a-j) show that Y_{low} BC overcomes T cell immunity and endows an immune suppressive TME. a, Tumor growth curves of Y+ and Y− MB49 grown subcutaneously in WT (n=8 mice per group), Ighm^{-/-} (n=10 and 7 mice per group, respectively), Tcrb^{-/-}/Tcrd^{-/-} (n=8 and 9 mice per group, respectively), or Rag2^{-/-} male mice (n=8 and 10 mice per group, respectively). Data are representative of two independent experiments. Statistical significance was determined by repeated measures two-way AVOVA. Data are mean±s.e.m. b, Uniform Manifold Approximation and Projection for Dimension Reduction (UMAP) map of spectral flow cytometry analysis of CD45⁺ immune cell subsets from Y+ and Y− MB49 tumors. c-d, Heatmap of UMAP immune cell populations (c) and individual protein markers (d) from Y+ and Y− MB49 TILs detailed in b. e, Volcano plot of statistically significant (p<0.05, green) UMAP clusters between Y+ and Y− MB49 tumors. Red and blue colors (and circles in c) denote Y− and Y+ associated clusters of interest, respectively. f, Violin plot of PD-1 and PD-L1 expression from Y− and Y+ MB49 CD45⁺ immune cells. Two-sided unpaired t-test. g, Volcano plot of statistically significant (red) GeoMX marker expression between Y+ and Y− MB49 tumors. h, Dot plot of mean gene expression (checkpoint molecules and markers of T cell activation/exhaustion) and fraction of cells from human MIBC CD45⁺ snSeq (single-nuclei RNA sequencing). i, Contour plot of T cells from human MIBC snSeq overlayed onto an annotated UMAP as described in Materials and Methods. Fold change enrichment between Y_{low} vs. Y_{high} MIBC T cell subsets. j, Stacked bar graph of immune cell subsets and T cell differentiation states from CODEX-stained Y_{low} vs. Y_{high} human MIBC tissue sections.

FIG. 4 (panels a-c) show Human Y_{low} BCs are enriched with CD8⁺ T cells with evidence of exhaustion. a, Violin plots of macrophages and CD8⁺ T cell immune scores from Y_{high} and Y_{low} BC using urothelial BC TCGA data (n=47 per group) and the Microenvironment Cell Populations-counter (MCP counter) method. Statistics conducted using two-sided Wilcoxon rank-sum test. b, Y signature score using snSeq data across 21 male naïve BC. Colors denote Y_{high} (blue, >0.2 mean Y signature score) and Y_{low} (red, <0.2 mean Y signature score) patient samples. c, Pie charts of the indicated non-epithelial immune cell types from samples described in b. d, Normalized expression of the indicated genes per T cell subtypes from MIBC samples identified in b. Y_{low} (red), Y_{high} (blue). T cells from melanoma and colon cancer samples were used to generate the referenced T cell subtypes and gene expression (black).

FIG. 5 (panels a-e) show superior response of Y_{low} BC to anti-PD-1 ICB therapy. a, Tumor growth curves of Y+ and Y− (top) and CRISPR-Y-Scr and CRISPR-Y-KO (bottom) treated with 200 g anti-PD-1 antibody or isotype control IgG. Y+ isotype and anti-PD1, n=17 mice per group. Y− isotype and anti-PD1, n=14 and 16 mice per group respectively. Data representative of two independent experiments. CRISPR-Y-Scr isotype and anti-PD1, n=11 mice per group. CRISPR-Y-KO isotype and anti-PD1, n=15 mice per group. Repeated measures two-way AVOVA. Data are mean s.e.m. b, UMAPs of CD8⁺ T cell spectral flow cytometry results from Y+ and Y− MB49 tumors seven days after treatments described in a. The UMAPs report cluster analyses (top) and cell proportions (bottom). c, UMAP heatmaps of individual protein markers in relation to b. d, Volcano plot of statistically significant (green) UMAP clusters between isotype and anti-PD-1 treated Y+ and Y− MB49 tumors. e, Kaplan-Meier curves of Y_{high} and Y_{low} anti-PD-L1-treated BC patients from the IMvigor210 dataset. Survival differences based on LogRank statistics.

FIG. 6 (panels a-d) shows increased genomic instability in Y_{low} BC. a, Heatmap of the indicated pathways and associated metadata from the IMvigor210 anti-PDL-1 clinical trial. b, Stacked bar graph of PD-L1 expression in immune cells (IC, left panel) and tumor cells (TC, right panel) from Y_{high} and Y_{low} BC samples identified in a. Pearson's Chi-squared test. c, Box plot of Y_{low} (n=67) and Y_{high} (n=129) neoantigen burden per megabase. Statistical significance was determined by Wilcoxon rank-sum test. d, Genomic instability pathway enrichment scores from BC described in a. Statistical significance was determined by Wilcoxon rank-sum test (Y_{low} n=33, Y_{high} n=82). APM, antigen-processing machinery. EMT, epithelial-to-mesenchymal transition. CR, complete response. PD, progressive disease. SD, stable disease. PR, partial response. IC, immune cells. TC, tumor cells. PD-L1 IHC scores, IC0 (<1%), ICI (>1% and <5%), or IC2/3 (>5%). Recomb, recombination, Nuc, nucleotide. Boxplots represent the mean with first and third quartile data. Minimum and maximum datapoints are included.

FIG. 7 (panels a-d) shows that LOY is associated with a worse clinical outcome for patients with MIBC and NMIBC. a, Y chromosome genes expressed in normal bladder urothelium that were used to create a Y chromosome gene expression signature. b, Log rank p-values based on stratification by Y chromosome gene expression (normalized FPKM) on TCGA MIBC patient overall survival (OS). Genes resulting in statistically significant OS are plotted in panel c. NE, not expressed. c, Kaplan-Meier plots of OS from TCGA data for males with MIBC and either high or low KDM5D, TBL1Y, UTY (KDM6C), or ZFY expression. d, Kaplan-Meier survival curves stratified by the Y signature score or expression levels for UTY and KDM5D in NMIBC from the E-MTAB-4321 cohort. Survival differences are based on Logrank statistics. e, ChrY gene expression signature scores of TCGA data plotted with respect to extreme downregulation of chromosome Y (EDY, left panel) and Mosaic Alteration Detection for LOY (mLOY, right panel) levels. Statistical significance was determined by Wilcoxon rank-sum test (NoLOY n=151, LOY n=90, NoEDY n=165, EDY n=76). Boxplots represent the mean with first and third quartile data. Minimum and maximum datapoints are included.

FIG. 8 (panels a-d) shows the generation of Y+ and Y− BC models. a, Histogram representation of deferentially regulated genes (DEG) from Y+ vs. Y− MB49 RNAseq data per mouse chromosome. b, qRT-PCR analysis of Uty, Kdm5d, Eif3y, and Ddx3y expression in MB49 clones isolated from the parental MB49 compared to female murine breast cancer (E0771) and bladder cancer cells (NA13), and testis tissue. Curly brackets indicate the clonal lines used to generate the pooled Y+ and Y− MB49 sublines. c, qRT-PCR analysis of Uty and Kdm5d expression in the pooled Y+ and Y− sublines described in a. n=3 biological replicates. Data are mean s.e.m. d, Bar graph of sequencing depth for each chromosome after performing whole exome sequencing (WES) on DNA from the Parental, Y−, and Y+ MB49 cell lines.

FIG. 9 (panels a-c) shows that LOY has no effect on colony forming ability of BC in vitro. a, MB49 Y+ and Y− cells were grown in 0.4% agar for two weeks. Colonies were stained with Nitro-BT and quantified using ImageJ. Average colony number and area were determined from those with a diameter that exceeded 100 μm (n=4 biological replicates). Data representative of three independent experiments. Statistical significance was determined by two-sided unpaired t-test, P-value=0.722. Data are mean±s.e.m. b, In vitro cell proliferation (MTT cell viability) over a 6-8-day time course using three sets of genetically engineered MB49 cells: Y+, Y+ Kdm5d KO, Y+ Uty KO (left panel), Y−, Y− Kdm5d OE, Y− Uty OE (middle panel), and CRISPR-Y-Scr vs. CRISPR-Y-KO (right panel). n=3 biological replicates. Data are mean s.e.m. c, qRT-PCR analysis of Uty expression in MB49 clones isolated from the CRISPR-generated Y-KO and Y-Scr MB49 cell lines. Curly brackets indicate the clonal lines used to generate the pooled Y+ Control and Y− KO MB49 sublines. Representative immunofluorescence images of the CRISPR-generated Y-KO and Y-Scr MB49 cell lines. Scale bar, 150 μM.

FIG. 10 (panels a-c) shows increased lymphocyte activation in Y+ tumors. a, Volcano plot of DEGs from bulk RNA isolated from Y+ and Y− MB49 tumors grown in male WT mice. Blue (Y+ tumors) and red (Y− tumors) genes correspond to statistically significant (Benjamini-Hochberg method, P<0.05) genes that have a |>1 log₂| fold-change in expression. b, PCA of DEGs described in a. c, Gene ontology (GO) pathway enrichment score plots of statistically significant gene set enrichment analyses (GSEA) using DEGs from a. NES, normalized enrichment score.

FIG. 11 (panels a-f) shows the comprehensive immune phenotyping of tumor-infiltrating leukocytes (TILs) in Y+ and Y− MB49 tumors. a, UMAPs demonstrating individual spectral flow cytometry analysis of protein marker expression in CD45⁺ immune cells isolated from Y+ and Y− MB49 tumors grown in WT male mice. b, Heatmap of relative protein expression from immune cells described in a. c, Violin plots of each tumor sample across each cluster from the CD45⁺ immune cell UMAP (see FIG. 3b). d, Violin plot of PD-1 and PD-L1 mean fluorescence intensity in CD45⁺ immune cells from Y+ and Y− MB49 tumors. e, Representative dot plots and percentages of CD8⁺ and CD4⁺ T cells gated on total CD3+ T cells from CRISPR-Y-Scr (n=8) and CRISPR-Y-KO (n=9), MB49 tumors grown for 22 or 17 days, respectively, in male WT mice (left panels). Percentage of CD8⁻ T cells of total CD3⁺ T cells per tumor sample (right panel). f, Percentage of CD206⁺PDL1⁺ macrophages among total CD11b⁺F4/80⁺ macrophages from Control and Y KO MB49 tumors described in e. Statistics were determined using two-sided unpaired t-tests.

FIG. 12 (paneles a-b) shows GeoMX histological evaluation of infiltrating immune cells in Y− and Y+ MB49 tumors. a, Representative H&E image (left), immunofluorescence detection of nuclei (blue), cytokeratin (green), CD45⁺ immune cells (red) (middle), and associated computational digital profiling (right) to quantify markers shown in b. Scale bar, 125 μM. b, Quantification (log 2 fold change and P-value) of the markers listed between Y+ and Y− MB49 tumors (n=10 tumors per group and three TMA cores per tumor). Data representative of two independent experiments. Statistical significance was determined by two-sided unpaired t-test.

FIG. 13 (panes a-f) shows the characterization of tumor-infiltrating CD8⁺ T cells after PD-1 pathway blockade. a-c, Relative spectral flow protein expression (a), sample-level violin plots per cluster (b), and heatmap of individual targets per cluster (c) after 200 pg anti-PD-1 or isotype control IgG treatments for 7 days using CD8⁺ T cells from Y+ and Y− MB49 tumors. d, Representative dot plots and percentages of TOX and/or GZMB-expressing CD8⁺ T cells from CRISPR-Y-Scr and CRISPR-Y-KO MB49 tumors grown in male WT mice after 200 g anti-PD-1 or isotype control IgG treatments for 7 days. e-f, Percentage of PD1⁻TOX⁺ CD8⁺ T cells (e) and TOX⁻CD44⁺ (top panel) or TOX⁻ICOS⁺ (bottom panel) CD8⁺ T cells (f) from tumor samples described in d. See FIG. 5 for additional method details. Statistical significance was determined by two-sided unpaired t-test. Tests were conducted between isotype controls or between isotype controls and anti-PD1 treatment groups.

FIG. 14 (panes a-c) shows the DDR-related pathways in TCGA Y_{low} vs. Y_{high} BC. a, Heatmap of the indicated pathways and metadata from BC TCGA data. b-c, box plot of tumor neoantigen burden (TNB) per megabase (P=0.700) (b), and associated pathway enrichment levels (c) from Y_{high} and Y_{low} tumors described in FIG. 1a. Statistical significance was determined by Wilcoxon test (Y_{low} n=118 and Y_{high} n=182). Boxplots represent the mean with first and third quartile data. Minimum and maximum datapoints are included.

FIG. 15 shows the defective DDR pathway activation in Y− MB49 cells. Normalized enrichment scores of statistically significant GSEA GO pathways using DEGs from Y− vs. Y+ MB49 cell cultures. Purple color denotes DNA repair-related pathways enriched in Y− cells.

FIG. 16 shows elevated genomic instability in LOY, Uty KO, and Kdm5d KO MB49 lines. Genome instability pathway enrichment scores using RNA-seq data from control and genetically modified MB49 cell lines (Y+ and Y− cells, n=5 technical replicates. n=3 for all other cell lines). Two-sided unpaired t-test. Boxplots represent the mean with first and third quartile data. Minimum and maximum datapoints are included.

FIG. 17 shows the objective response rate (ORR) of various tumor types to anti-PD-1/PD-L1 therapy based on the expression level of Y signature based on the 18 genes listed FIG. 7B

FIG. 18 shows the objective response rate (ORR) of various tumor types to anti-PD-1/PD-L1 therapy based on the expression level of NLGN4Y.

FIG. 19 shows the objective response rate (ORR) of various tumor types to anti-PD-I/PD-L1 therapy based on the expression level of Y chromosome genes (BPY2, CDY1B, CYorf15A, Cyorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, and CDY1A).

FIG. 20 shows the expression of male-specific region on chromosome Y (MSY) genes in human tissues of the Human Protein Atlas RNA-Seq. Red blocks indicate expression >0.1 RPKM per gene per tissue or presence in immunohistocytochemistry. Genes colored red are shared between rodents (rat and mouse) and human, including genes identified to be species-specific duplication of a single gene in the other species, such as TSPY genes of human. Variants identified in multiple databases are shown to the right. Databases included ClinVar (www.ncbi.nlm.nih.gov/clinvar), dbSNP 142 for nonsynonymous (ns) and frameshift (fs) mutations, Catalog of Somatic Mutations in Cancer (COSMIC, cancer.sanger.ac.uk/cosmic), and the Human Gene Mutation Database (HGMD, www.hgmd.cf.ac.uk/) (prior art, see Prokop and Deschepper, Physiol Genomics. 2015 November; 47(11): 525-537).

FIG. 21 shows the overall survival of patients. (bottom line) Y-high group, the (top line) LOY group, both are treated with PD-i/PD-L1 inhibitors. The LOY group is more responsive, and thus, have higher survival.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects are intended to be including within the scope of this term.

“Therapeutically effective amount” as used herein refers to that amount which is capable of achieving beneficial results in a patient with cancer; for example, bladder cancer. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or lessen the disease even if the treatment is ultimately unsuccessful.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, pen-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions may be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions may be in the form of aerosol, lotion, cream, gel, ointment, suspensions, solutions or emulsions. In accordance with the present disclosure, “administering” may be self-administering. For example, it is considered as “administering” that a subject consumes a composition as disclosed herein.

By “at risk of” is intended to mean at increased risk of, compared to a normal subject, or compared to a control group, e.g. a patient population. Thus a subject carrying a particular marker may have an increased risk for a specific condition, disease or disorder, and be identified as needing further testing. “Increased risk” or “elevated risk” mean any statistically significant increase in the probability, e.g., that the subject has the disorder. In some embodiments the risk is increased by at least 10% over the control group with which the comparison is being made. In some embodiments, the risk is increased by at least 20% over the control group with which the comparison is being made. In some embodiments, the risk is increased by at least 50% over the control group with which the comparison is being made.

“Sample” is used herein in its broadest sense. The term “biological sample” as used herein denotes a sample taken or isolated from a biological organism. Exemplary biological samples include, but are not limited to, cheek swab; mucus; whole blood, blood, serum; plasma; urine; saliva; semen; lymph; fecal extract; sputum; other body fluid or biofluid; cell sample; and tissue sample etc. The term also includes a mixture of the above-mentioned samples. The term “sample” also includes untreated or pretreated (or pre-processed) biological samples. In some embodiments, a sample can comprise one or more cells from the subject. In some embodiments, a sample is a tissue or tissue sample. In some embodiments, the sample is a tumor or tumor sample or tumor tissue.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal may be a human, non-human primate, mouse, rat, dog, cat, horse, or cow, but are not limited to these examples. In addition, the methods described herein may be used to treat domesticated animals and/or pets. In some embodiments, the subject is a human. In some embodiments, the subject is a male subject. In some embodiments, the subject has a cancer. In some embodiments, the subject has a tumor.

Extreme downregulation of chromosome Y (EDY) is a male-specific signature of cancer susceptibility that is strongly linked to LOY and Y chromosome methylation patterns. Given that BC is the fourth most common cancer in men and that LOY occurs in up to 40% of cases, regardless of grade and stage, the understanding of how LOY contributes to the poor prognoses of males is an unmet medical need and a major biological question. Described herein, we show that aggressive behavior of LOY BC is a consequence of T cell dysfunction. We report increased tumor-associated macrophages, high levels of immune checkpoint molecules, and CD8 T cell exhaustion in Y_{low} tumors. Consistent with known mechanisms of response to immunotherapy, patients with Y_{low} tumors exhibit a superior response to anti-PD-1/PD-L1 ICB. We coin the term LOYalT to highlight the phenomenon of LOY in cancer in altering T cell function.

We first examined TCGA BC RNAseq data using a Y chromosome gene expression signature and found that patients with LOY had a reduced overall survival following surgery. Given that the MB49 murine BC model is a widely used model to investigate different aspects of BC biology and has also been shown by others to lose the Y chromosome, we used MB49 to isolate two distinct, naturally arising cell populations to represent Y_{low} and Y_{high} tumors. Consistent with human data, MB49 tumors with LOY grew more aggressively. Similar results were seen when knocking out two chromatin modifying genes located on the Y chromosomes, UTY and KDM5D. Overexpressing these genes in Y− lines regained tumor control in the immune competent host. Loss of UTY, the male counterpart of UTX (KDM6A) located on the X chromosome, has been reported to promote BC development. Unlike UTX, UTY possesses very low demethylase activity for H3K27, suggesting that UTY suppresses BC development in a demethylase-independent manner. On the other hand, KDM5D negatively regulates expression of genes involved in tumor cell invasion, such as the matrix metalloproteinase (MMP) family, by demethylation of their H3K4me3 marks. Downregulation of KDM5D expression increases H3K4me3 levels at target gene promoter regions, leading to a more aggressive phenotype and the development of metastasis.

Through use of mouse models lacking T cells (Tcrb/Tcrc^{-/-}), B cells (Ighm^{-/-}) or both (Rag2^{-/-}Il2rg^{-/-}, Rag2^{-/-}), we demonstrated that the differential growth observed between Y− and Y+ MB49 tumors was T cell-dependent. Multispectral flow cytometric analysis of these tumors, as well as in depth analyses of human MIBC specimens through snSeq and histological applications, revealed that CD8⁺ T cells within the Y+ tumors retained their anti-tumor immunity, whereas CD8⁺ T cells in Y− tumors were phenotypically exhausted. Of significance, TOX, a transcription factor typically known to transcriptionally and epigenetically program CD8⁺ T cells towards terminal exhaustion, was elevated in both CD8^{+ and CD}4⁻ T cells at various differentiation states within Y_{low} BC specimens. The molecular mechanism of LOYalT is unclear. In particular, how loss of Y chromosome genes from tumor cells leads to upregulation of TOX in tumor-infiltrating T cells and its role in BC progression warrants further investigation.

Importantly, our findings with LOY cancer variants were completely recapitulated with CRISPR/Cas9 mediated chromosome depletion of Y+ cells, providing strong evidence that the LOYalT phenomenon was driven entirely by loss of Y chromosome, not by any cryptic clonal variations. In essence, we discovered that LOY tumors were able to evade adaptive immunity by promoting CD8⁺ T cell exhaustion. By doing so, LOY tumors were also more responsive to anti-PD-1 ICB because the primary mode of ICB is driving T cell differentiation from exhaustion to effector function. Taken together, evaluation of Y chromosome gene expression, especially UTY and KDM5D, could be used to select BC patients for ICB therapy with the expectation of superior response and better survival outcomes. Additionally, transient pharmacologic inhibition of UTY and KDM5D may offer enhanced therapeutic benefit for Y_{high} patients undergoing ICB therapy.

Accordingly, various embodiments of the present invention are based, at least in part, on these findings.

Various embodiments of the invention provide for a method of selecting a cancer treatment for a male subject, comprising: assaying a biological sample obtained from the male subject to determine a Y chromosome gene expression signature, detecting a loss of Y chromosome (LOY) phenotype, and selecting a therapy comprising an immune checkpoint inhibitor, a TOX inhibitor, or a combination thereof, or detecting a Y chromosome high phenotype, and selecting a therapy comprising an immune checkpoint inhibitor and an inhibitor of one or more genes on the Y chromosome.

In various embodiments, the cancer is selected from colon adenocarcinoma microsatellite instability (COAD MSI), skin cutaneous melanoma (SKCM), kidney renal clear cell carcinoma (KIRC), bladder cancer (BLCA), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD). In particular embodiments, the cancer is bladder cancer.

In various embodiments, detecting LOY phenotype comprises detecting a low level of one or more genes on the Y chromosome, as compared to each gene's reference level. Examples of those genes are described herein. Detection of two or more genes, three or more genes, four or more genes, five or more genes, ten or more gene, 15 or more genes, 18 or more genes, 20 or more gene, 25 or more genes, 30 or more genes, or 35 or more genes on the Y chromosome are also contemplated as being part of the invention. Similarly, the detection of 1-5 genes, 6-10 genes, 11-15 genes, 16-20 genes, 21-25 genes, 26-30 genes, 31-35 genes on the Y chromosome are also contemplated as being part of the invention. As a further example, the detection of 18 genes on the Y chromosome is contemplated as being part of this invention. Additional examples are detection of 12 genes on the Y chromosome. Additional examples are detection of 3 genes on the Y chromosome. Additional examples are detection of 29 genes on the Y chromosome.

In various embodiments, the one or more genes being detected on the Y chromosome is selected from BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the one or more genes being detected on the Y chromosome is selected from BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the one or more genes being detected on the Y chromosome is selected from DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, or ZFY. In various embodiments, the one or more genes being detected on the Y chromosome are DAZ1, DAZ2, DAZ3, DAZ4, DDX3Y, EIF1AY, HSFY2, KDM5D, NLGN4Y, PCDH11 Y, PRY2, RBMY1A1, RPS4Y1, TBL1Y, TMSB4Y, USP9Y, UTY, and ZFY.

In various embodiments, the one or more genes being detected on the Y chromosome is KDM5D, or UTY (KDM6C), or both. In various embodiments, the one or more genes being detected on the Y chromosome is selected from KDM5D, UTY (KDM6C), TBL1Y, or ZFY. In various embodiments, the one or more genes being detected on the Y chromosome is NLGN4Y.

In various embodiments, the one or more genes being detected on the Y chromosome comprise KDM5D, UTY (KDM6C), TBL1Y, ZFY, DDX3Y, USP9Y, RPS4Y1, TMSB4Y, EIF1AY, NLGN4Y, HSFY2, PCDH11Y, SRY, HSFY1, PRY2, DAZ1, DAZ3, DAZ4, DAZ2, RPS4Y2, RBMY1A1, BPY2, BPY2B, AMELY, TSPY3, TSPY8, RBMY1B, RBMY1E, BPY2C, TGIF2LY, TSPY2, TSPY4, TSPY1, TSPY10, BPY1, BPY1B, CDY2B, CDY2A, CDY1B, CDY1A or a combination thereof.

Each gene's reference level can be the expression level in a normal tissue source of the cancer. For example, for bladder cancer, each gene's reference level can be the expression level in a normal male bladder tissue (see e.g., Prokop and Deschepper, Physiol Genomics. 2015 November; 47(11): 525-537).

In various embodiments detecting LOY phenotype comprises detecting Y signature score of less than 0.2. In various embodiments detecting LOY phenotype comprises detecting Y signature score of less than 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30. In various embodiments detecting LOY phenotype comprises detecting Y signature score of less than 0.1. In various embodiments detecting LOY phenotype comprises detecting Y signature score of less than 0.15. In various embodiments detecting LOY phenotype comprises detecting Y signature score of less than 0.25. In various embodiments detecting LOY phenotype comprises detecting Y signature score of less than 0.3. Calculation of the Y signature score can be done as described herein.

In various embodiments, the inhibitor to the one or more genes on the Y chromosome is an inhibitor to one or more genes selected from BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the inhibitor to the one or more genes on the Y chromosome is an inhibitor to one or more genes selected from BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the inhibitor to the one or more genes on the Y chromosome is an inhibitor to one or more genes selected from DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, or ZFY. In various embodiments, the one or more genes being detected on the Y chromosome are DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, and ZFY.

In various embodiments, the inhibitor to the one or more genes on the Y chromosome is an inhibitor to one or more genes selected from KDM5D, UTY (KDM6C, or both. In various embodiments, the inhibitor to the one or more genes on the Y chromosome is an inhibitor to one or more genes selected from KDM5D, UTY (KDM6C), TBL1Y, or ZFY. In various embodiments, the inhibitor to the one or more genes on the Y chromosome is an inhibitor to NLGN4Y.

In various embodiments, the inhibitor to the one or more genes on the Y chromosome is an inhibitor to one or more genes selected from KDM5D, UTY (KDM6C), TBL1Y, ZFY, DDX3Y, USP9Y, RPS4Y1, TMSB4Y, EIF1AY, NLGN4Y, HSFY2, PCDH11Y, SRY, HSFY1, PRY2, DAZ1, DAZ3, DAZ4, DAZ2, RPS4Y2, RBMY1A1, BPY2, BPY2B, AMELY, TSPY3, TSPY8, RBMY1B, RBMY1E, BPY2C, TGIF2LY, TSPY2, TSPY4, TSPY1, TSPY10, BPY1, BPY1B, CDY2B, CDY2A, CDY1B, or CDY1A.

Examples of inhibitors to one or more of these gene include but are not limited to antisense oligonucleotides, shRNAs, or siRNAs to these one or more genes.

In various embodiments, the biological sample is a tumor sample. In particular embodiments, the sample is a bladder cancer sample.

In various embodiments, the immune checkpoint inhibitor is an anti-PD-L1 inhibitor or an anti-PD-1 inhibitor.

Examples of PD1 inhibitor include pembrolizumab, nivolumab, pidilizumab, AMP-224, AMP-514, spartalizumab, cemiplimab, penpulimab (AK105), prolgolimab (BCD-100), ezabenlimab (BI 754091), toripalimab (JS001), lipustobart (LZM009), retifanlimab (MGA012), Sym021, dostarlimab (TSR-042), tebotelimab (MGD013), cadonilimab (AK104), vudalimab (XmAb20717), tislelizumab, PF-06801591, anti-PD1 antibody expressing pluripotent killer T lymphocytes (PIK-PD-1), and autologous anti-EGFRvIII 4SCAR-IgT cells. Examples of PDL1 inhibitor include garivulimab (BGB-A333), cosibelimab (CK-301), FAZ053, envafolimab (KN035), MDX-1105, betifisolimab (MSB2311), adebrelimab (SHR-1316), atezolizumab, avelumab, durvalumab, BMS-936559, CK-301, and M7824.

In various embodiments, the immune checkpoint inhibitor is atezolizumab, avelumab, durvalumab, nivolumab, or pembrolizumab.

In various embodiments, the immune checkpoint inhibitor is atezolizumab. In various embodiments, the immune checkpoint inhibitor is pembrolizumab. In various embodiments, the immune checkpoint inhibitor is avelumab.

In various embodiments, the method further comprises administering the selected therapy.

Various embodiments of the invention provide for a method of treating cancer in a male subject, comprising: administering a therapy comprising an immune checkpoint inhibitor, a TOX inhibitor, or a combination thereof to the male subject, wherein the male subject has been detected to have a loss of Y chromosome (LOY) phenotype, or administering a therapy comprising immune checkpoint inhibitor and an inhibitor of one or more genes selected on the Y chromosome, to the male subject, wherein the male subject has been detected to have a Y chromosome high phenotype.

In various embodiments, the cancer is selected from colon adenocarcinoma microsatellite instability (COAD MSI), skin cutaneous melanoma (SKCM), kidney renal clear cell carcinoma (KIRC), bladder cancer (BLCA), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma PRAD. In particular embodiments, the cancer is bladder cancer.

In various embodiments, the inhibitor to the one or more genes on the Y chromosome is an inhibitor to one or more genes selected from BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the inhibitor to the one or more genes on the Y chromosome is an inhibitor to one or more genes selected from BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the inhibitor to the one or more genes on the Y chromosome is an inhibitor to one or more genes selected from DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, or ZFY. In various embodiments, the one or more genes being detected on the Y chromosome are DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, and ZFY.

In various embodiments, the inhibitor to the one or more genes on the Y chromosome is an inhibitor to one or more genes selected from KDM5D, UTY (KDM6C), or both. In various embodiments, the inhibitor to the one or more genes on the Y chromosome is an inhibitor to one or more genes selected from KDM5D, UTY (KDM6C), TBL1Y, or ZFY. In various embodiments, the inhibitor to the one or more genes on the Y chromosome is an inhibitor to NLGN4Y.

In various embodiments, the inhibitor to the one or more genes on the Y chromosome is an inhibitor to one or more genes selected from KDM5D, UTY (KDM6C), TBL1Y, ZFY, DDX3Y, USP9Y, RPS4Y1, TMSB4Y, EIF1AY, NLGN4Y, HSFY2, PCDH11Y, SRY, HSFY1, PRY2, DAZ1, DAZ3, DAZ4, DAZ2, RPS4Y2, RBMY1A1, BPY2, BPY2B, AMELY, TSPY3, TSPY8, RBMY1B, RBMY1E, BPY2C, TGIF2LY, TSPY2, TSPY4, TSPY1, TSPY10, BPY1, BPY1B, CDY2B, CDY2A, CDY1B, or CDY1A.

Examples of inhibitors to one or more of these gene include but are not limited to antisense oligonucleotides, shRNAs, or siRNAs to these one or more genes.

In various embodiments, the immune checkpoint inhibitor is an anti-PD-L1 inhibitor or an anti-PD-1 inhibitor.

Examples of PD1 inhibitor include pembrolizumab, nivolumab, pidilizumab, AMP-224, AMP-514, spartalizumab, cemiplimab, penpulimab (AK105), prolgolimab (BCD-100), ezabenlimab (BI 754091), toripalimab (JS001), lipustobart (LZM009), retifanlimab (MGA012), Sym021, dostarlimab (TSR-042), tebotelimab (MGD013), cadonilimab (AK104), vudalimab (XmAb207l7), tislelizumab, PF-06801591, anti-PD1 antibody expressing pluripotent killer T lymphocytes (PIK-PD-1), and autologous anti-EGFRvIII 4SCAR-IgT cells. Examples of PDL1 inhibitor include garivulimab (BGB-A333), cosibelimab (CK-301), FAZ053, envafolimab (KN035), MDX-1105, betifisolimab (MSB2311), adebrelimab (SHR-1316), atezolizumab, avelumab, durvalumab, BMS-936559, CK-301, and M7824.

In various embodiments, the immune checkpoint inhibitor is atezolizumab, avelumab, durvalumab, nivolumab, or pembrolizumab.

In various embodiments, the immune checkpoint inhibitor is atezolizumab. In various embodiments, the immune checkpoint inhibitor is pembrolizumab. In various embodiments, the immune checkpoint inhibitor is avelumab.

Various embodiments of the invention provide for a method of treating cancer in a male subject, comprising: requesting the results of an assay of a biological sample obtained from the male subject to determine a Y chromosome gene expression signature; administering a therapy comprising an immune checkpoint inhibitor, a TOX inhibitor, or a combination thereof to the male subject, wherein the male subject has been detected to have a loss of Y chromosome (LOY) phenotype, or administering a therapy comprising immune checkpoint inhibitor and an inhibitor of one or more genes on the Y chromosome to the male subject, wherein the male subject has been detected to have a Y chromosome high phenotype.

In various embodiments, the cancer is selected from colon adenocarcinoma microsatellite instability (COAD MSI), skin cutaneous melanoma (SKCM), kidney renal clear cell carcinoma (KIRC), bladder cancer (BLCA), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma PRAD. In particular embodiments, the cancer is bladder cancer.

In various embodiments, detecting LOY phenotype comprises detecting a low level of one or more genes on the Y chromosome, as compared to each gene's reference level. Detection of two or more genes, three or more genes, four or more genes, five or more genes, ten or more gene, 15 or more genes, 18 or more genes, 20 or more gene, 25 or more genes, 30 or more genes, or 35 or more genes on the Y chromosome are also contemplated as being part of the invention. Similarly, the detection of 1-5 genes, 6-10 genes, 11-15 genes, 16-20 genes, 21-25 genes, 26-30 genes, 31-35 genes on the Y chromosome are also contemplated as being part of the invention. As a further example, the detection of 18 genes on the Y chromosome is contemplated as being part of this invention. As a further example, the detection of 18 genes on the Y chromosome is contemplated as being part of this invention. Additional examples are detection of 12 genes on the Y chromosome. Additional examples are detection of 3 genes on the Y chromosome. Additional examples are detection of 29 genes on the Y chromosome.

In various embodiments detecting LOY phenotype comprises detecting Y signature score of less than 0.2. In various embodiments detecting LOY phenotype comprises detecting Y signature score of less than 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30. In various embodiments detecting LOY phenotype comprises detecting Y signature score of less than 0.1. In various embodiments detecting LOY phenotype comprises detecting Y signature score of less than 0.15. In various embodiments detecting LOY phenotype comprises detecting Y signature score of less than 0.25. In various embodiments detecting LOY phenotype comprises detecting Y signature score of less than 0.3. Calculation of the Y signature score can be done as described herein.

In various embodiments, the one or more genes being detected on the Y chromosome is selected from BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1AI, RPS4Y2, KDM5D, TMSB4Y, USP9Y, UTY, XKRY, BPY1, or CDY1A.

In various embodiments, the one or more genes being detected on the Y chromosome is selected from BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY

In various embodiments, the one or more genes being detected on the Y chromosome is selected from DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, or ZFY. In various embodiments, the one or more genes being detected on the Y chromosome are DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, and ZFY.

In various embodiments, the one or more genes being detected on the Y chromosome is KDM5D, or UTY (KDM6C), or both. In various embodiments, the one or more genes being detected on the Y chromosome is selected from KDM5D, UTY (KDM6C), TBL1Y, or ZFY. In various embodiments, the one or more genes being detected on the Y chromosome is NLGN4Y.

In various embodiments, the one or more genes being detected on the Y chromosome comprise KDM5D, UTY (KDM6C), TBL1Y, ZFY, DDX3Y, USP9Y, RPS4Y1, TMSB4Y, EIF1AY, NLGN4Y, HSFY2, PCDH11Y, SRY, HSFY1, PRY2, DAZ1, DAZ3, DAZ4, DAZ2, RPS4Y2, RBMY1A1, BPY2, BPY2B, AMELY, TSPY3, TSPY8, RBMY1B, RBMY1E, BPY2C, TGIF2LY, TSPY2, TSPY4, TSPY1, TSPY10, BPY1, BPY1B, CDY2B, CDY2A, CDY1B, CDY1A or a combination thereof.

Each gene's reference level can be the expression level in a normal tissue source of the cancer. For example, for bladder cancer, each gene's reference level can be the expression level in a normal male bladder tissue (see e.g., Prokop and Deschepper, Physiol Genomics. 2015 November; 47(11): 525-537).

In various embodiments, the inhibitor to the one or more genes on the Y chromosome is an inhibitor to one or more genes selected from BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the inhibitor to the one or more genes on the Y chromosome is an inhibitor to one or more genes selected from BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the inhibitor to the one or more genes on the Y chromosome is an inhibitor to one or more genes selected from DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, or ZFY. In various embodiments, the one or more genes being detected on the Y chromosome are DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, and ZFY.

In various embodiments, the inhibitor to the one or more genes on the Y chromosome is an inhibitor to one or more genes selected from KDM5D, UTY (KDM6C), or both. In various embodiments, the inhibitor to the one or more genes on the Y chromosome is an inhibitor to one or more genes selected from KDM5D, UTY (KDM6C), TBL1Y, or ZFY. In various embodiments, the inhibitor to the one or more genes on the Y chromosome is an inhibitor to NLGN4Y.

In various embodiments, the inhibitor to the one or more genes on the Y chromosome is an inhibitor to one or more genes selected from KDM5D, UTY (KDM6C), TBL1Y, ZFY, DDX3Y, USP9Y, RPS4Y1, TMSB4Y, EIF1AY, NLGN4Y, HSFY2, PCDH11Y, SRY, HSFY1, PRY2, DAZ1, DAZ3, DAZ4, DAZ2, RPS4Y2, RBMY1A1, BPY2, BPY2B, AMELY, TSPY3, TSPY8, RBMY1B, RBMY1E, BPY2C, TGIF2LY, TSPY2, TSPY4, TSPY1, TSPY10, BPY1, BPY1B, CDY2B, CDY2A, CDY1B, or CDY1A.

Examples of inhibitors to one or more of these gene include but are not limited to antisense oligonucleotides, shRNAs, or siRNAs to these one or more genes.

In various embodiments, the biological sample is a tumor sample. In particular embodiments, the sample is a bladder cancer sample.

In various embodiments, the immune checkpoint inhibitor is an anti-PD-L1 inhibitor or an anti-PD-I inhibitor.

Examples of PD1 inhibitor include pembrolizumab, nivolumab, pidilizumab, AMP-224, AMP-514, spartalizumab, cemiplimab, penpulimab (AK105), prolgolimab (BCD-100), ezabenlimab (BI 754091), toripalimab (JS001), lipustobart (LZM009), retifanlimab (MGA012), Sym021, dostarlimab (TSR-042), tebotelimab (MGD013), cadonilimab (AK104), vudalimab (XmAb207l7), tislelizumab, PF-06801591, anti-PD1 antibody expressing pluripotent killer T lymphocytes (PIK-PD-1), and autologous anti-EGFRvIII 4SCAR-IgT cells. Examples of PDL1 inhibitor include garivulimab (BGB-A333), cosibelimab (CK-301), FAZ053, envafolimab (KN035), MDX-1105, betifisolimab (MSB2311), adebrelimab (SHR-1316), atezolizumab, avelumab, durvalumab, BMS-936559, CK-301, and M7824.

In various embodiments, the immune checkpoint inhibitor is atezolizumab, avelumab, durvalumab, nivolumab, or pembrolizumab.

In various embodiments, the immune checkpoint inhibitor is atezolizumab. In various embodiments, the immune checkpoint inhibitor is pembrolizumab. In various embodiments, the immune checkpoint inhibitor is avelumab.

Various embodiments of the invention provide for a method of determining susceptibility to treatment with an immune checkpoint inhibitor, a TOX inhibitor, or both, in a male subject having cancer, comprising assaying a biological sample obtained from the male subject to determine a Y chromosome gene expression signature; detecting a loss of Y chromosome (LOY) phenotype, wherein the LOY indicates a susceptibility treatment with an immune checkpoint inhibitor, a TOX inhibitor, or both, or detecting a Y chromosome high phenotype, wherein the Y chromosome high phenotype indicates a low likelihood of susceptibility treatment with an immune checkpoint inhibitor, a TOX inhibitor, or both, or indicates a benefit from a therapy comprising an immune checkpoint inhibitor and an inhibitor of one or more genes in the Y chromosome.

In various embodiments, the cancer is selected from colon adenocarcinoma microsatellite instability (COAD MSI), skin cutaneous melanoma (SKCM), kidney renal clear cell carcinoma (KIRC), bladder cancer (BLCA), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma PRAD. In particular embodiments, the cancer is bladder cancer.

In various embodiments, detecting LOY phenotype comprises detecting a low level of one or more genes on the Y chromosome, as compared to each gene's reference level. Detection of two or more genes, three or more genes, four or more genes, five or more genes, ten or more gene, 15 or more genes, 18 or more genes, 20 or more gene, 25 or more genes, 30 or more genes, or 35 or more genes on the Y chromosome are also contemplated as being part of the invention. Similarly, the detection of 1-5 genes, 6-10 genes, 11-15 genes, 16-20 genes, 21-25 genes, 26-30 genes, 31-35 genes on the Y chromosome are also contemplated as being part of the invention. As a further example, the detection of 18 genes on the Y chromosome is contemplated as being part of this invention. As a further example, the detection of 18 genes on the Y chromosome is contemplated as being part of this invention. Additional examples are detection of 12 genes on the Y chromosome. Additional examples are detection of 3 genes on the Y chromosome. Additional examples are detection of 29 genes on the Y chromosome.

In various embodiments detecting LOY phenotype comprises detecting Y signature score of less than 0.2. In various embodiments detecting LOY phenotype comprises detecting Y signature score of less than 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30. In various embodiments detecting LOY phenotype comprises detecting Y signature score of less than 0.1. In various embodiments detecting LOY phenotype comprises detecting Y signature score of less than 0.15. In various embodiments detecting LOY phenotype comprises detecting Y signature score of less than 0.25. In various embodiments detecting LOY phenotype comprises detecting Y signature score of less than 0.3. Calculation of the Y signature score can be done as described herein.

In various embodiments, the one or more genes being detected on the Y chromosome is selected from BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the one or more genes being detected on the Y chromosome is selected from BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY

In various embodiments, the one or more genes being detected on the Y chromosome is selected from DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, or ZFY. In various embodiments, the one or more genes being detected on the Y chromosome are DAZ1, DAZ2, DAZ3, DAZ4, DDX3Y, EIF1AY, HSFY2, KDM5D, NLGN4Y, PCDH11 Y, PRY2, RBMY1A1, RPS4Y1, TBL1Y, TMSB4Y, USP9Y, UTY, and ZFY.

In various embodiments, the one or more genes being detected on the Y chromosome is KDM5D, or UTY (KDM6C), or both. In various embodiments, the one or more genes being detected on the Y chromosome is selected from KDM5D, UTY (KDM6C), TBL1Y, or ZFY. In various embodiments, the one or more genes being detected on the Y chromosome is NLGN4Y.

In various embodiments, the one or more genes being detected on the Y chromosome comprise KDM5D, UTY (KDM6C), TBL1Y, ZFY, DDX3Y, USP9Y, RPS4Y1, TMSB4Y, EIF1AY, NLGN4Y, HSFY2, PCDH11Y, SRY, HSFY1, PRY2, DAZ1, DAZ3, DAZ4, DAZ2, RPS4Y2, RBMY1A1, BPY2, BPY2B, AMELY, TSPY3, TSPY8, RBMY1B, RBMY1E, BPY2C, TGIF2LY, TSPY2, TSPY4, TSPY1, TSPY10, BPY1, BPY1B, CDY2B, CDY2A, CDY1B, CDY1A or a combination thereof.

Each gene's reference level can be the expression level in a normal tissue source of the cancer. For example, for bladder cancer, each gene's reference level can be the expression level in a normal male bladder tissue (see e.g., Prokop and Deschepper, Physiol Genomics. 2015 November; 47(11): 525-537).

In various embodiments, the inhibitor to the one or more genes on the Y chromosome is an inhibitor to one or more genes selected from BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

In various embodiments, the inhibitor to the one or more genes on the Y chromosome is an inhibitor to one or more genes selected from BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY.

In various embodiments, the inhibitor to the one or more genes on the Y chromosome is an inhibitor to one or more genes selected from DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, or ZFY. In various embodiments, the one or more genes being detected on the Y chromosome are DAZ1, DAZ2, DAZ3, DAZ4, DDX3Y, EIF1AY, HSFY2, KDM5D, NLGN4Y, PCDH11Y, PRY2, RBMY1A1, RPS4Y1, TBL1Y, TMSB4Y, USP9Y, UTY, and ZFY.

In various embodiments, the inhibitor to the one or more genes on the Y chromosome is an inhibitor to one or more genes selected from KDM5D, UTY (KDM6C), or both. In various embodiments, the inhibitor to the one or more genes on the Y chromosome is an inhibitor to one or more genes selected from KDM5D, UTY (KDM6C), TBL1Y, or ZFY. In various embodiments, the inhibitor to the one or more genes on the Y chromosome is an inhibitor to NLGN4Y.

In various embodiments, the inhibitor to the one or more genes on the Y chromosome is an inhibitor to one or more genes selected from KDM5D, UTY (KDM6C), TBL1Y, ZFY, DDX3Y, USP9Y, RPS4Y1, TMSB4Y, EIF1AY, NLGN4Y, HSFY2, PCDH11Y, SRY, HSFY1, PRY2, DAZ1, DAZ3, DAZ4, DAZ2, RPS4Y2, RBMY1A1, BPY2, BPY2B, AMELY, TSPY3, TSPY8, RBMY1B, RBMY1E, BPY2C, TGIF2LY, TSPY2, TSPY4, TSPY1, TSPY10, BPY1, BPY1B, CDY2B, CDY2A, CDY1B, or CDY1A.

Examples of inhibitors to one or more of these gene include but are not limited to antisense oligonucleotides, shRNAs, or siRNAs to these one or more genes.

In various embodiments, the biological sample is a tumor sample. In particular embodiments, the sample is a bladder cancer sample.

In various embodiments, the immune checkpoint inhibitor is an anti-PD-L1 inhibitor or an anti-PD-1 inhibitor.

Examples of PD1 inhibitor include pembrolizumab, nivolumab, pidilizumab, AMP-224, AMP-514, spartalizumab, cemiplimab, penpulimab (AK105), prolgolimab (BCD-100), ezabenlimab (BI 754091), toripalimab (JS001), lipustobart (LZM009), retifanlimab (MGA012), Sym021, dostarlimab (TSR-042), tebotelimab (MGD013), cadonilimab (AK104), vudalimab (XmAb20717), tislelizumab, PF-06801591, anti-PD1 antibody expressing pluripotent killer T lymphocytes (PIK-PD-1), and autologous anti-EGFRvIII 4SCAR-IgT cells. Examples of PDL1 inhibitor include garivulimab (BGB-A333), cosibelimab (CK-301), FAZ053, envafolimab (KN035), MDX-1105, betifisolimab (MSB2311), adebrelimab (SHR-1316), atezolizumab, avelumab, durvalumab, BMS-936559, CK-301, and M7824.

In various embodiments, the immune checkpoint inhibitor is atezolizumab, avelumab, durvalumab, nivolumab, or pembrolizumab.

In various embodiments, the immune checkpoint inhibitor is atezolizumab. In various embodiments, the immune checkpoint inhibitor is pembrolizumab. In various embodiments, the immune checkpoint inhibitor is avelumab.

### Y Signature Score

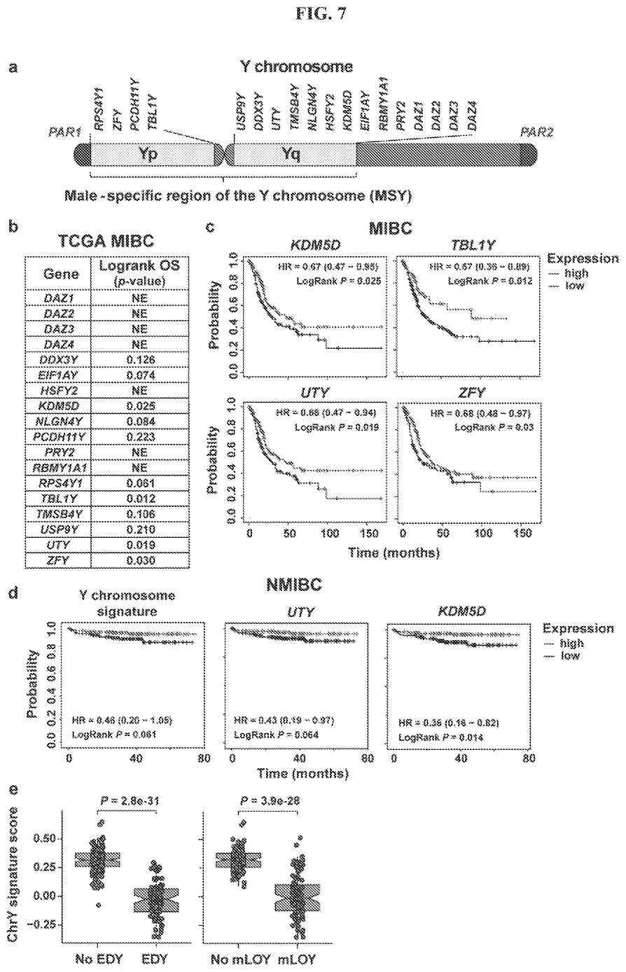

As an example, a gene expression signature score was created based on 18 Y-encoded genes expressed in normal bladder urothelium (DAZ1, DAZ2, DAZ3, DAZ4, DDX3Y, EIF1AY, HSFY2, KDM5D, NLGN4Y, PCDH11Y, PRY2, RBMY1AI, RPS4Y1, TBL1Y, TMSB4Y, USP9Y, UTY, ZFY (see FIG. 7B)). To obtain the Y signature consisting of 18 important Y chromosome genes expressed in each of the bulk RNA-seq samples from TCGA data, IMvigor210 cohorts, and E-MTAB-4321 cohort. We utilized the single sample Gene Set Enrichment Analysis (ssGSEA) method using the GSEApy package (v0.10.1) (Hanzelmann et al., GSVA: gene set variation analysis for microarray and RNA-seq data. BMC Bioinformatics 14, 7, doi:10.1186/1471-2105-14-7 (2013)). ssGSEA is a non-parametric method that calculates a gene set enrichment score per sample as the normalized difference in empirical cumulative distribution functions (CDFs) of gene expression ranks inside and outside the gene set. The implementation in the GSVA package normalizes pathway scores by dividing them by the range of calculated values, following the last step described in Barbie et al., Systematic RNA interference reveals that oncogenic KRAS-driven cancers require TBK1. Nature 462, 108-112 (2009). For the snRNA-seq dataset, we used the Scanpy (scanpy.tl.score_genes function; scanpy v1.9.1) (Wolf et al. SCANPY: large-scale single-cell gene expression data analysis. Genome Biol 19, 15, doi:10.1186/s13059-017-1382-0 (2018)) to get the Y signature for each patient. The score is the average expression of a set of genes subtracted by the average expression of a reference set of genes which is randomly sampled from the gene pool for each binned expression value.

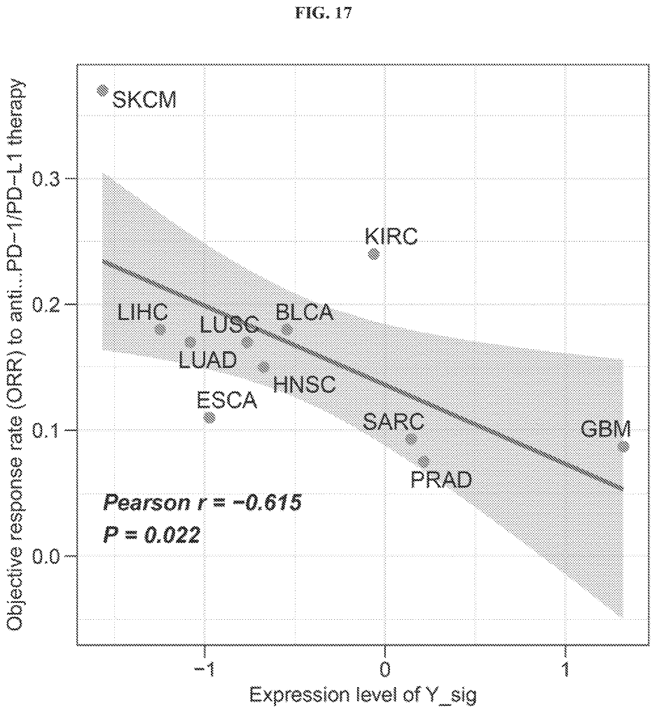

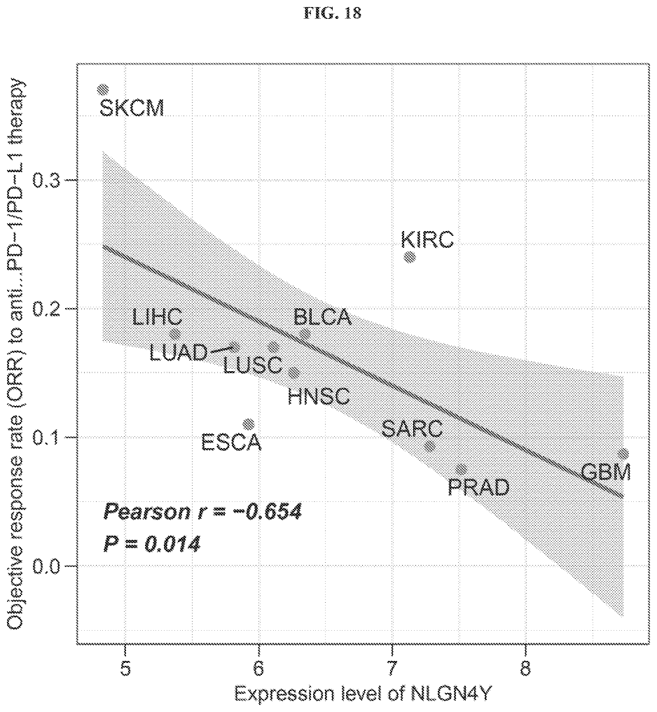

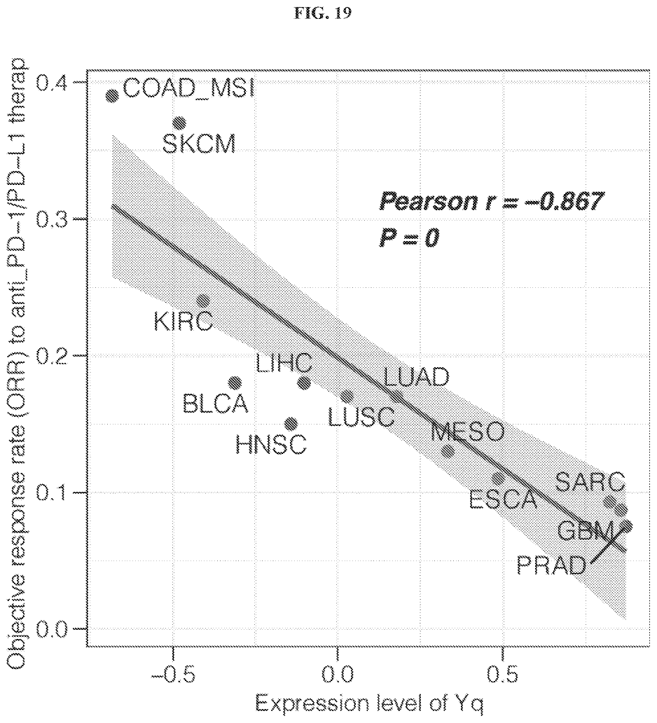

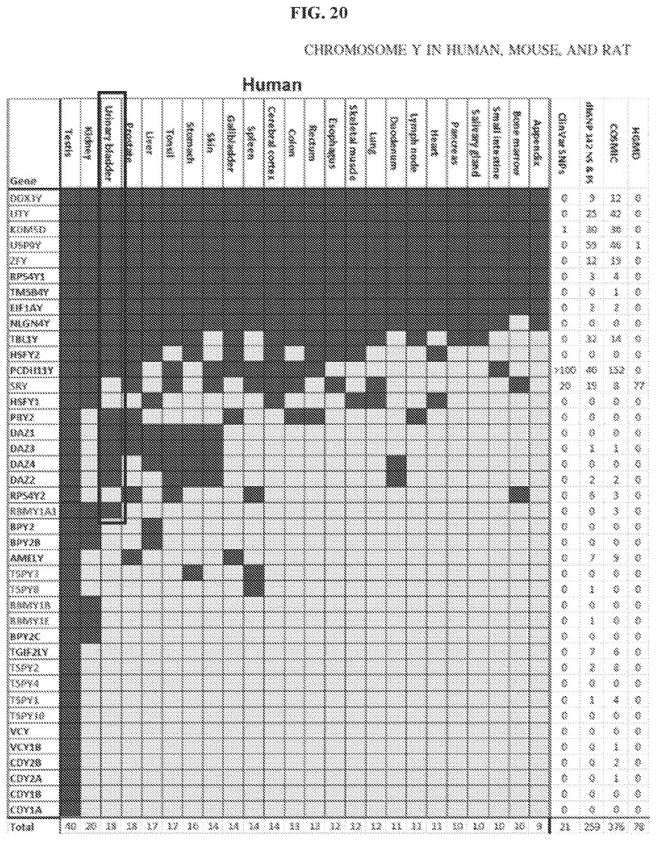

This was also applied this signature on a broad range of data used whole-exome and RNA sequencing of 7187 patients from the publicly available Cancer Genome Atlas and the objective response rate (ORR) data to anti-PD-1/PD-1 therapy of 21 cancer types obtained from a collection of clinical trials. Use of the LOY signature had a remarkable significant trend across all cancer types (FIGS. 17 and 19) while individual analysis of genes found NLGN4Y (FIG. 18) as the gene whose expression was most strongly associated with response.

While the gene expression signature score was created based on 18 Y-encoded genes expressed in normal bladder urothelium, additional gene expression signatures and cognate scores can be created based on various Y-encoded genes expressed in corresponding normal tissue (for use in other types of tumors). As a nonlimiting example, 12 Y-encoded genes (or any other number of Y-encoded genes) expressed in normal (non-cancerous) lung tissue can be used to generate a gene expression signature score using the methodology described above and then using the gene expression signature score for determining LOY in lung cancers. Another nonlimiting example includes the use of Y-encoded genes expressed in any normal tissue to develop a pan-cancer signature that correlates with outcome following immune checkpoint blockade as shown in FIG. 19.

According, in an exemplary form, the following steps can be used to determine a gene signature scope:
Step 1. Selection of Geneset: 18 Y-encoded genes were chosen (for this example).
Step 2. Data Sources:

Bulk RNA-seq samples from TCGA data, IMvigor210 cohorts, and the E-MTAB-4321 cohort were used to obtain the Y signature. SnRNA-seq data from nature communications paper.
Step 3. Analysis Method:

For bulk RNA-seq samples: Single sample gene set enrichment analysis (ssGSEA) was performed using the GSEApy package (version 0.10.1). This analysis was conducted for each sample to determine the expression levels of the 18 Y chromosome genes.

For snRNA-seq dataset: Scanpy package (version 1.9.1) was utilized, specifically the ‘scanpy.tl.score_genes’ function, to compute the Y signature for each patient.
Step 4. Result:

The gene expression signature score for each patient was obtained, reflecting the expression levels of the 18 Y-encoded genes in their respective datasets.

This methodology allows for the quantification of the Y chromosome gene expression signature in different datasets and enables comparisons across samples.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Materials and Methods

### Cell Culture

The parental MB49 cell line (from University of Virginia). Cell lines were authenticated morphology and whole exome sequencing. All cell lines tested negative for mycoplasma contamination. The MB49 Y− and Y+ sublines were cultured in DMEM supplemented with 10% fetal bovine serum (FBS) and 0.1% sodium pyruvate. NA13 cell line was isolated from N-butyl-N-(4-hydroxybutyl) nitrosamine (BBN) carcinogen-induced bladder tumors of C57BL/6 female mice and cultured in RPMI with 10% FBS. The E0771 breast cancer cell line E0771 (from University of Colorado) was cultured in RPMI supplemented with 5% FBS. Single cell isolating of MB49 Y− and Y+ cells were performed by serial dilution in 96 well plates.

### In Vivo Tumor Growth

Experiments were carried out at both Cedar-Sinai Medical Center and The Ohio State University under Institutional Animal Care & Use Committee (IACUC) approved protocols. Six-eight week old male WT C57BL/6 and various syngeneic immuno-deficient mice were purchased from Taconic or the Jackson Laboratory, and kept in a specific pathogen-free environment. Bladder cancer cell lines were detached, washed with PBS, and resuspended to make 10⁶ cells per ml in phenol red-free DMEM media and left on ice. Animals were anaesthetized by isoflurane and cells were injected into the flank of each mouse subcutaneously (10⁴ cells in 100pl for MB49 sublines). The sample size in each experimental group was determined based on a previously published studies of similar models. Mice were randomized to treatment groups and blinded to researcher for the duration of the experiment. The same batch and number of cancer cells were injected per experiment, and all animals were housed under the same conditions. Investigators were blinded to group allocation during the experiment. Tumor length and width were measured using calipers, and tumor volume was calculated by (L×W²)/2, where L is the largest diameter measurement of the tumor and W is the shortest perpendicular tumor measurement. All analysis was performed consistently during all studies and all tumor counts were performed by the same investigator. For experiments involving PD-I blockade, mice were treated with anti-PD-1 antibody (200 pg, BioXCell clone 29F.1A12X) or isotype control IgG (200 g) every three days starting when tumors reached ~100 mm³.

### Isolation of TILs and High Dimensional Flow Cytometry

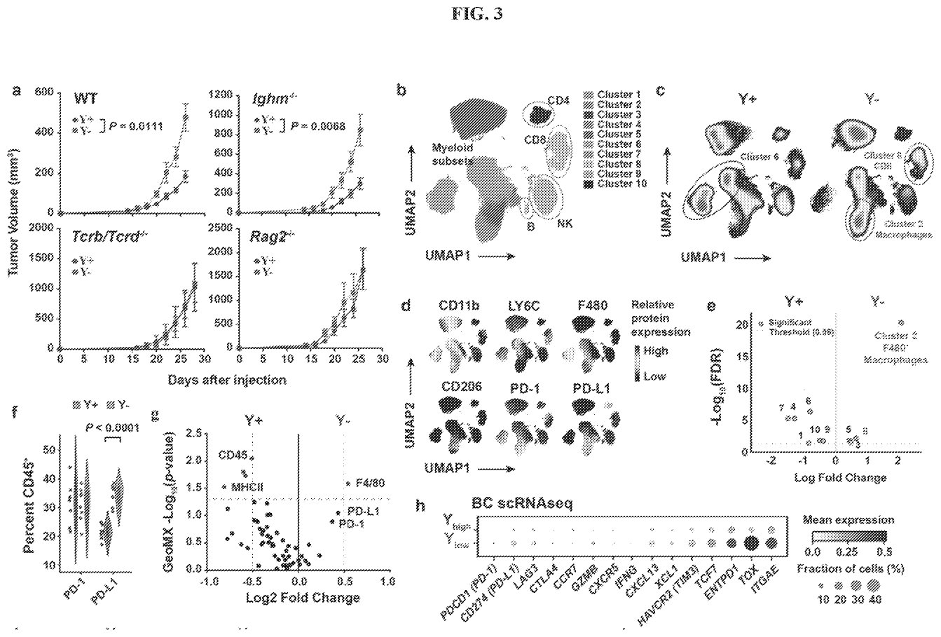

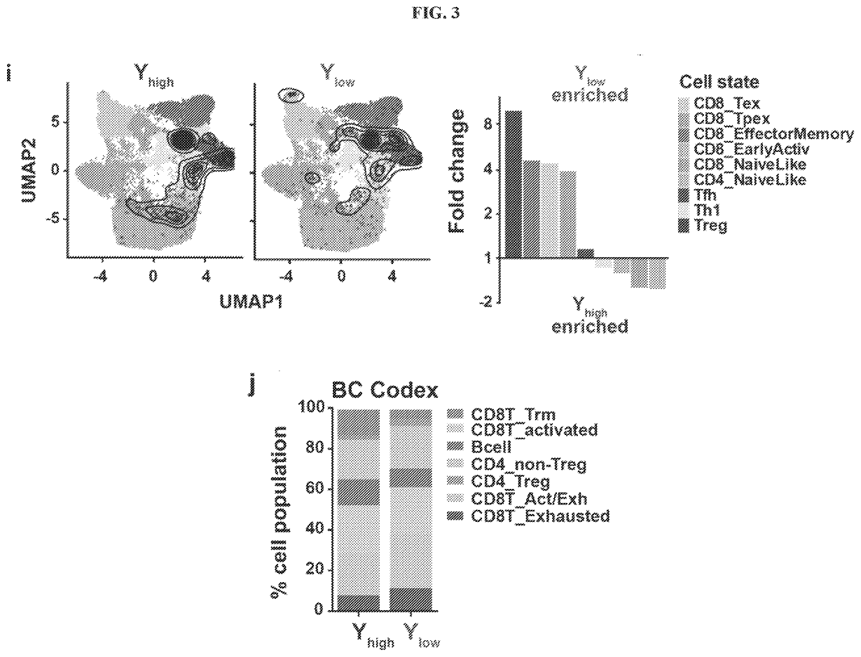

Endpoint Y+ and Y−, as well as CRISPR-Y-Scr and CRISPR-Y-KO, MB49 tumors were mechanically disrupted and subjected to digestion via 1 mg/mL Collagenase D (Roche) for 30 min at 37 °C with agitation. Two percent bovine serum albumin in ice-cold PBS was used to inactivate enzymatic activity. Red blood cell lysis buffer (BioLegend) was used before cells were passed through 70 μm filters to generate single cell suspensions. Cells were then stained at 4 °C with LIVE/DEAD blue fixable viability dye for 15 minutes (Invitrogen), followed by extracellular surface markers and FcR block concurrently for 40 minutes. All intracellular staining was performed using the FoxP3 transcription factor staining kit (Invitrogen) according to the manufacturer's instructions. All results were acquired on the Cytek Aurora, and fluorochrome-conjugated antibody panels were applied as previously described. To analyze spectral flow cytometry results, live CD45 singlets were gated from samples stained with the ‘All Immune Phenotyping’ flow antibody panel (FIG. 3) and live CD45⁺ CD11b⁻CD4⁻CD8⁺CD3⁺ singlets were gated from samples stained with the ‘T Cell Exhaustion’ panel (FIG. 5) using OMIQ to generate the presented UMAPs and heatmaps.

### In Vitro Cell Proliferation and Colony Formation Assays

Cell proliferation of MB49 sublines was determined using methyl thiazolyl diphenyl-tetrazolium bromide (MTT, Sigma). Different cell numbers (200-25,000) were seeded into 96-well plates with 150 μl of medium per well. Cells were assessed daily over an 8-day time course by adding MTT solution (1.25 mg/ml in DMEM) to cultures at a 1:1 ratio and incubating for two hours at 37 °C. Lysis buffer was added to dissolve the formazan crystals. Absorbance was read at 570 nm by a plate reader (BioTeK). MTT assays were performed three times in triplicate for each cell line tested. To check if Y chromosome specific genes were involved in anchorage-independent growth, soft agar colony formation assays were performed. Briefly, 15,000 cells/well in a 0.4% agar (Sigma) were plated in triplicate in 6-well plates and cultured at 37 °C for two weeks. Colonies were stained with Nitro-BT (Sigma), and the images of the plates were captured under a microscope and analyzed using ImageJ software.

### Total RNA Extraction and Quantitative Real-Time PCR

RNA was extracted from subcutaneous Y+ and Y− tumors or cell lysate using a RNeasy plus Mini Kit with gDNA Eliminator (Qiagen). Library preparation and RNA sequencing was performed by Novogene (Sacramento, CA). cDNA was synthetized using Maxima H Minus cDNA Synthesis Master Mix (Thermo Scientific). qPCR was then performed using Quant Studio 6 Flex Real-Time PCR system (Applied Biosystems) via powerUp SYBR Green Master Mix (Applied Biosystems). Forward and reverse primer pairs used in qRT-PCR experiments are listed in the table below. To determine the changes in mRNA expression measured by qRT-PCR, the ΔΔCₜ method was used. p-actin served as an internal control.

| | | SEQ | | SEQ |
|---|---|---|---|---|
| Gene | | ID | | ID |
| name | Forward primer | NO | Reverse primer | NO |
| Gapdh | AACTTTGGCATTGTGGAAGG | 1 | ACACATTGGGGGTAGGAAC | 2 |
| | | | A | |
| β-Actin | TCATGAAGTGTGACGTTGAC | 3 | CCTAGAAGCATTTGCGGTGC | 4 |
| | ATCCGT | | ACGATG | |
| Kdm5d | CAT | 5 | ATGAATGCGTCAGATTGGG | 6 |
| | GTAAAGGAGATAAGGAACT | | | |
| Sry | TCC TCA AAA GAA ACC GTG | 7 | AGATTAATGGTTGCTAAGGA | 8 |
| | CAT | | CTGGAT | |
| Ssty1 | CTGGAGCTCTACAGTGATGA | 9 | CAGTTACCAATCAACACATC | 10 |
| | | | AC | |
| Eif2s3y | ATCTTGTCCTCAACCTCAGA | 11 | TTCTTTAGCCTGGCTTTCTTT | 12 |
| | CT | | CA | |
| Uty | TGGCCCTGATCGACTGTAAT | 13 | TGGTGCATCCAACCTAACTG | 14 |
| Ddx3y | TGTTAGTTGCCACACCAGGA | 15 | TGGTGGCATTGTGTCCTGCT | 16 |
| | CG | | CA | |
| Rbmy | TGCTGGCTATCTTGGAGGAC | 17 | GCGGTTGTTTCCACCTCTTG | 18 |
| | GT | | CA | |

### RNA Sequencing Data Analysis (RNAseq)

Illumina Truseq adapter, polyA, and polyT sequences were trimmed with cutadapt v2.3. Trimmed reads were aligned to mouse genome version 38 (mm10) using STAR aligner v2.7.0d_0221 with parameters according to the ENCODE long RNA-seq pipeline (github.com/ENCODE-DCC/long-rna-seq-pipeline). Gene expression levels were quantified using RSEM v1.3.1. Ensembl gene annotations (version 84) were used in the alignment and quantification steps. RNA-seq sequence, alignment, and quantification quality was assessed using FastQC v0.11.5 and MultiQC v1.8. Biological replicate concordance was assessed using PCA and pairwise Pearson correlation analysis. Low expressed genes were filtered out by applying the following criterion: estimated counts (from RSEM) >number of samples*5. Filtered estimated read counts from RSEM were compared using the R Bioconductor package DESeq2 v1.22.2 based on generalized linear model and negative binomial distribution. Genes with Benjamini-Hochberg corrected p-value <0.05 and fold change >1.5 or <1.5 were selected as differentially expressed. Differentially expressed genes were then analyzed using the Ingenuity Pathway Analysis (Qiagen).

Engineering of UTY and KDM5D expression in MB49 cells

CRISPR/Cas9 editing of UTY and KDM5D in Y+ MB49 cells: CRISPR/Cas9 KO plasmids were purchased from Santa Cruz. UTY CRISPR/Cas9 KO Plasmid (m2) as a pool of 3 different gRNA plasmids: A—Sense: TCTTTAATAGTAAAAGCTGA (SEQ ID NO:19) (sc-423636-KO-2); B—Sense: GATGAAGACGCTGTTGAACA (SEQ ID NO:20) (sc-423636-KO-2); and C—Sense: ACAGTTTACAGACTGACTAC (SEQ ID NO:21) (sc-423636-KO-2). KDM5D (SmcY) CRISPR/Cas9 KO Plasmid (m) is a pool of three different gRNA plasmids: A—Sense: GGACCTTTACAGCCTTAATA (SEQ ID NO:22) (sc-423031); B—Sense: TTCAGTTGCTACAGTAGACG (SEQ ID NO:23) (sc-423031); and C—Sense: TCATTTAGCCTCTGAATTCG (SEQ ID NO:24) (sc-423031). The control was a non-targeting gRNA (not targeting any known gene; sc-418922). CRISPR/Cas9 plasmids were transfected into Y+ MB49 cells using UltraCrus transfection reagent according to the manufacturer's instruction (Santa Cruz). Forty-eight hours after transfection, the medium was aspirated and replaced with fresh medium containing puromycin for selection. For validation of KO cells, single cells were isolated as described above. RNA from each clone (using a RNeasy kit) was used to validate KO of UTY or KDM5D.

Overexpression of UTY and KDM5D in MB49 Y− cells: Plasmid ps100001 from Origene was used to overexpress KDM5D and UTY. Empty vector served as the negative control. Lipofectamine 3000 (Thermo Fisher) was used for all transfections according to the manufacturer's protocol. Forty-eight hours after transfection, the medium was replaced with fresh medium containing neomycin for selection.

### Genomic DNA Isolation and Whole Exome Sequencing (WES)

Genomic DNA was isolated from parental, Y−, and Y+ MB49 cell lines by Invitrogen Kit (catalog No K1820) according to the manufacturer's instructions. DNA samples were submitted for sequencing at Novogene (Sacramento, CA). Library preparation, sequencing, and bioinformatics analysis were performed by Novogene. Briefly, all samples were sent for WES and the sequencing data processed using the GATK Best Practice workflow. The genomic DNA was randomly sheared into short fragments, end-repaired, A-tailed, and ligated with Illumina adapters. After PCR amplification, size selection, and purification, we proceeded with the hybridization capture of libraries. The captured libraries were enriched by PCR amplification and checked with Qubit and bioanalyzer for quality control. The libraries were then pooled and sequenced on Illumina platforms using the PE150 strategy. We mapped the paired-end clean reads to the mouse reference genome (Mouse, NCBI37/mm9) using BWA, sorted them with Sambamba, and marked duplicates with Picard. Finally, we computed the coverage and depth, and tested for SNP and InDel detection.

### Generation of the CRISPR/Cas9-Mediated Y Chromosome KO MB49

Plasmids: Single guide RNA (gRNA) targeting the centromere of the Y chromosome (LOY_gRNA1_fwd CACCGGAGTTAATATAAAAAACA (SEQ ID NO:25) and LOY_gRNA1_rv AAACTGTTTTTTATATTAACTCC (SEQ ID NO:26)) alongside the non-targeting control gRNA (Control_(Neo)_gRNA1_fwd CACCGGCAGCGCGGCTATCGTGGC (SEQ ID NO:27) and Control_(Neo)_gRNA1_rv AAACGCCACGATAGCCGCGCTGCC (SEQ ID NO:28)) were a gift from Dr. Kenneth Walsh (University of Virginia, Charlottesville, VA, USA). LentiCRISPRv2 neo, Cas9 Plasmid #98292; pLKO5.0.sgRNA.EFS.tRFP, plasmid #57823; psPAX2; plasmid #12260; and pMD2.G, plasmid #12259 plasmids were purchased from Addgene.

Lentivirus production: HEK 293T cells were seeded at a density of 4×10⁶ cells per 10 cm² plate in DMEM+5% FBS and incubated at 37 °C, 5% CO₂. The plasmids (5 μg of lentiCRISPRv2 neo or pLKO5.0.sgRNAs.EFS.tRFP, 4.17 μg psPAX2, and 0.83 g of pMD2.G per plate) were co-transfected using HEK293T cells with Trans-IT transfection reagent (Mirus). Culture supernatant was collected 48 h after transfection, centrifuged, and filtered through a 0.45 μm filter. Given that the LOY-gRNA plasmid also contains a turbo red fluorescent protein (tRFP) marker, we sorted the cells based on tRFP 48 hours after transduction into 96 wells using BD Symphony S6 cell sorter for further culturing. Single cell clones were expanded and screened for KO of the Y chromosome by qRT PCR. Control cells were generated by transducing Cas9 Y+ MB49 cells with control gRNA.

### Nanostring GeoMx Digital Spatial Profiling (DSP)

H&E images of Y+ and Y− MB49 tumors were demarcated by a clinical pathologist (Cedars-Sinai Biobank Core) for regions in each core to generate a tumor microarray (TMA). Five-micron sections were cut from the TMA block, stained, and analyzed on the Nanostring GeoMx Digital Spatial Profiling (DSP) platform at the Cedars-Sinai Biobank Core as per manufacturer's instructions. The protein panel consisted of 68 antibodies, including mouse immune cell profiling, immuno-oncology (IO) drug targets, immune activation, immune cell typing, pan-tumor, and cell death markers (FIG. 10A). S6 ribosomal protein and histone 3 were included in the panel as housekeeping proteins.

One region of interest (ROI) per core was selected by aligning fluorescent images with H&E images predetermined by a pathologist. Regions with blood vessels and necrosis were kept to a minimum. ROI selection on each TMA core was performed such that 660 μm circles were segmented into cytokeratin+ (tumor) and cytokeratin-(stroma) regions. Barcodes from these regions were collected to generate measurements per compartment. Barcodes were sequenced, mapped, and counted by next-generation sequencing (NGS) readouts as per manufacturer's instructions. Antibody barcodes were counted on an Ncounter platform as per manufacturer's instructions allowing quantitative comparisons of antibodies between ROIs in Y+ and Y− subcutaneous tumors.

The GeoMx DSP analysis suite (GEOMX-0069) allowed inspection, quality control (QC), normalization, and differential expression to be performed. Briefly, normalization using Histone H3 and S6 proteins was performed. Differential expression between paired compartments was evaluated by paired t-tests with a Benjamini-Hochberg correction, while differential expression between unpaired compartments was performed by a Mann-Whitney test with Benjamini-Hochberg correction. GraphPad prism software version 8.0 (GraphPad Prism) was used for all of the statistical analyses. The student's two tailed t-test was used to analyze all comparisons, with a Bonferroni adjustment for multiple comparisons when appropriate.

### Y Signature Score

We created a gene expression signature score was created based on 18 Y-encoded genes expressed in normal bladder urothelium as previously reported (FIG. 7B). The Y signature was obtained by the single sample Gene Set Enrichment Analysis (ssGSEA, package GSEApy v0.10.1) according to 18 important Y chromosome genes expressed in each of the bulk RNA-seq samples from TCGA data, IMvigor210 cohorts, and E-MTAB-4321 cohort. For the snRNA-seq dataset, we used the Scanpy (scanpy.tl.score_genes function; scanpy v1.9.1) to get the Y signature for each patient. The score is the average expression of a set of genes subtracted by the average expression of a reference set of genes which is randomly sampled from the gene pool for each binned expression value. Y chromosome signature scores were compared to two virtual karyotyping techniques, EDY using TCGA RNA data and mLOY using TCGA DNA sequencing data.

### Gene Set Enrichment Analysis (GSEA)

Genes differentially expressed in specific sample groups were identified using DESeq2 software (version 1.26.0) with the Wald test by using a design formula that included batch effect correction. Benjamini-Hochberg correction was used for multiple testing in all instances. We performed GSEA (GSEApy v0.10.1) to identify significantly differentially enriched pathways between the Y+ and Y− MB49 cell lines and tumors. The gene ontology gene set in the MSigDB database was used as the reference gene set. The default weighted enrichment statistical method was applied to the enrichment analysis, and the number of the gene set permutations for each analysis was set at 1,000. Statistical significance was set at a |normalized enrichment score|>1, nominal P-value <0.05.

### Human Bladder Cancer RNA-Seq Datasets

We performed differential gene expression analyses on publicly available urothelial bladder cancer datasets from the IMvigor210 clinical trial, TCGA, and NMIBC E-MTAB-4321 datasets. For the analysis involving IMvigor210, only male pre-chemotherapy bladder cancer samples were used. Objective response rate, important signature score, and survival data for each male patient was extracted using the R package Imvigor210CoreBiologies. For the TCGA analysis, the data levels, and the files contained in each data level package, are described below and are present on the NCI Genomic Data Commons. We estimated cell population abundance in the TCGA MIBC samples using the microenvironment cell populations-counter (MCP counter) method, which uses transcriptomic data to deconvolute and quantify the abundance of various immune cell types. For the snSeq analyses, 21 treatment-naïve MIBC male patients, with surgery (TURBT/cystectomy) as their only treatment, can be downloaded from GEO (GSE169379, GSE171351) in our previous study. To explore this transcriptional heterogeneity, we classified single cells using the “ProjecTILs” T cell atlas derived from tumor-infiltrating T lymphocytes (TILs). The atlas consists of 16,803 high-quality single-cell transcriptomes from 25 samples (B16 melanoma and MC38 colon adenocarcinoma tumors) from six different studies.

### Co-Detection by Indexing (CODEX)

We obtained our patient-matched co-detection by indexing (CODEX) data from our same bladder cancer snRNA-seq study, Processed CODEX data can be downloaded from: figshare.com/s/461Oa15363c8306dfa36, figshare.com/s/2005255a8b65de23109f, figshare.com/s/1d8c7ed76d4b3222ada4.

The CODEX technology allows for highly multiplexed analysis for up to 40 proteins using cyclic detection of DNA-indexed antibody panels. In this study, CODEX was grouped according to Y signature, which was obtained based on the snRNA-seq data of 21 male bladder cancer patients. Seven patients with high scores were selected as the Y_{high} group (>0.2 mean Y signature score) and 7 patients with low scores as Y_{low} group (<0.2 mean Y signature score). CODEX-stained cells were manually gated based on the intensity of PanCytoK, CD45, aSMA, CD31, CD20, CDH12, CDH18, CD68, CD3e, CD8, and CD4 into a training set consisting of broad cell types: Epithelial, Epithelial KRT, Epithelial CDH, Stromal, Endothelial, General CD45⁺ immune, Bcell, CD8T, CD4T, and Macrophage.

### Kaplan-Meier Analyses

Clinical outcomes were assessed using Kaplan-Meier survival curves and Log-Rank/Cox statistics. Kaplan-Meier survival analysis was performed using the survminer package in R software. The function “surv_cutpoint” in the R package “survminer” was implemented to discover the optimal cut-off value for the Y chromosome signature. This R package determines the optimal cut-point for one or multiple continuous variables at once, using the maximally selected rank statistics from the ‘maxstat’ R package. This is an outcome-oriented method providing a value of a cut-point that corresponds to the most significant relation with survival.

### Quantification of Tumor Neoantigen Burden

For each patient in the IMvigor210 and TCGA databases, we first enumerated a list of all possible 9 and 10-mer peptides bearing somatic mutations or overlapping open reading frames derived from frameshifting indels or nonstop mutations. These peptides were then evaluated for binding against the patient's inferred HLA type using the NetMHCpan-3.0 algorithm. The neoantigen load was defined as the total number of predicted peptide:allele binders with a rank percentile score less than or equal to the weak binder threshold (2%).

### Statistical Analyses

The statistical tests employed, the number of replicates, and independent experiments are specified in the text and figure legends. Basic statistical analysis was performed using GraphPad/Prism (v.9.0), while R (versions 4.1.2 or 4.2.3) and Python 3.9.4 were utilized for analyses of epidemiological and mutation/sequence data. Prior to conducting any statistical test, a Kolmogorov-Smirnov normality test was performed, and Bartlett or Levene tests were used to evaluate the assumption of homogeneity of variances. The unpaired Student's t-test was applied to normally distributed variables, while the Mann-Whitney U test (also known as the Wilcoxon rank sum test) was employed for non-normally distributed variables.

Pearson's Chi-squared test was used for analyzing categorical data, with the R function ‘chisq.test’ utilized for chi-squared analyses and ‘wilcox.test’ for Wilcoxon rank sum tests. Significance levels were denoted using asterisks, with “NS” indicating non-significance. In cases where multiple hypotheses were tested, FDR correction was applied. Data analysis software such as Scanpy (v.1.7.2), Pandas (v.2.0.0), Conda (v.4.11.0), NumPy (v.1.24.2), and Scipy (v.1.10.1) were used. The R package ComplexHeatmap (v.2.11.1) was employed for generating heat maps, while ggplot2 (v.3.3.5), ggpubr (v.0.6.0), and ggrepel (v.0.9.2) R packages were used for general visualization purposes.

### Example 2

### Results

LOY is Associated with a Worse Outcome for Patients with Locally Advanced BC

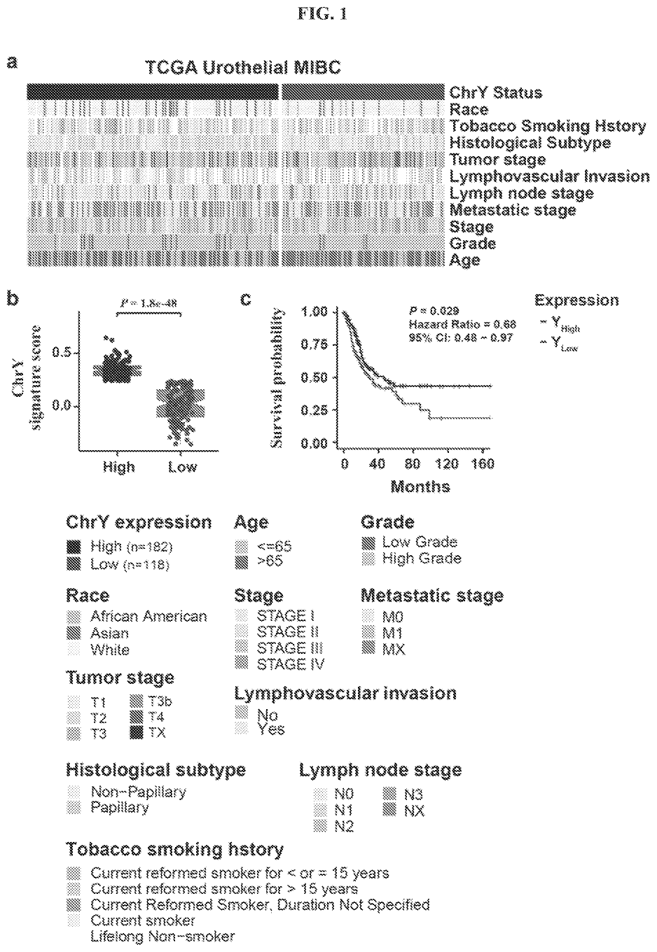

We created a “Y chromosome” RNA expression signature score based on 18 Y-encoded genes expressed in normal bladder urothelium (FIG. 7a-b). We used this score to stratify the overall survival of 300 men with locally advanced/muscle-invasive bladder cancer (MIBC) found in TCGA transcriptomic data. Based on average Y chromosome signature scores, male samples were divided into two groups: Y_{high} (N=182) and Y_{low} (N=118) (FIG. 1a-b). Patients' age (34-90 years), stage (tumor, lymph node, metastatic lesion), race (white, African American, Asian), or tumor grade (high, low) were similar between Y_{high} vs Y_{low} samples (FIG. 1a). However, patients with low Y chromosome gene expression score had significantly (P=0.024) worse overall survival compared to those with higher expression (FIG. 1c). Importantly, decreased individual expression of 4 Y chromosome genes [KDM5D, UTY (KDM6C), TBL1Y, ZFY]was also associated with a poor prognosis (FIG. 7c). Similar survival results were found when 834 patients with non-muscle-invasive bladder cancer (NMIBC) were evaluated (FIG. 7d), suggesting that LOY is present early in disease progression.

To confirm the robustness of results obtained with our Y chromosome signature score, we also carried out virtual karyotyping analyses using two different recently published techniques. The first study used TCGA RNA data to show that extreme downregulation of chromosome Y (EDY) increased cancer risk in 12 cancer types, including bladder cancer. The second study used TCGA DNA sequencing data, and a technique named Mosaic Alteration Detection for LOY (MADloy aka mLOY) to detect LOY from genotype-array-intensity data. Use of these two techniques validated our Y chromosome signature as Y_{low} tumor specimens were also identified as having EDY and mLOY and suggests low Y chromosome gene expression is due to the loss of the Y chromosome (FIG. 7e).

### Development of Y Chromosome Positive and Negative Murine BC Models

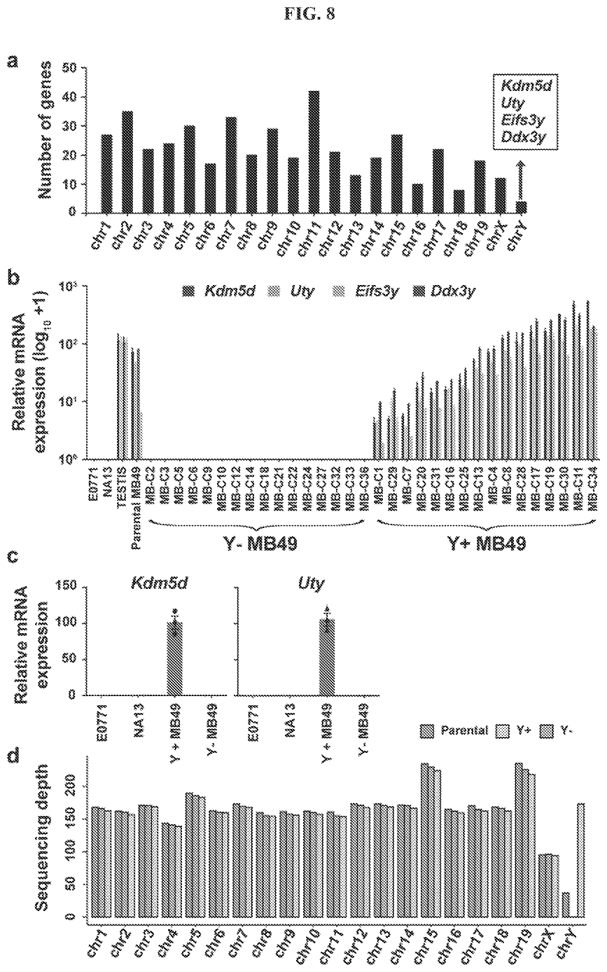

To determine why Y_{low} BC is more aggressive than Y_{high}, tumors in patients, we assessed Y chromosome gene expression in MB49, a well-studied murine BC cell line that has been shown to naturally lose the Y chromosome. We selected and expanded single-cell clones from the heterogeneous MB49 parental population and assessed the expression of seven genes located on the male-specific region on the chromosome Y (MSY) that are commonly expressed in both mouse and human bladder cells. Only three (Kdn5d, Uty, and Ddx3y) of the seven genes were expressed in the MB49 line (FIG. 8a and Table 1). Eif2s3y was also evaluated as it is an additional mouse Y chromosome gene known to be expressed in urothelium. Sixteen clones that exhibited no Y chromosome gene expression compared to positive controls (normal murine testis and the parental MB49 line) were deemed as Y negative and pooled together to create a polyclonal Y negative subline. NA13 and E0771 are female bladder and breast cancer cell lines, respectively, and served as negative controls. Similarly, 16 clones with expression of the four Y chromosome genes, comparable to positive controls, were considered Y positive and pooled to create a single Y positive line (FIG. 8b-c). Next, by performing whole exome sequencing (WES), we confirmed that the lack of Y chromosome gene expression in the MB49 sublines was due to LOY (FIG. 8d). Henceforth, we will refer to these lines as Y positive (Y+) and Y negative (Y−).

TABLE 1
Y chromosome gene expression in MB49 clones

| Cell line | kdm5d | Uty | Ddx3y | Eifs3y | Sstyl | Sry | Rbmy |
|---|---|---|---|---|---|---|---|
| E0771 | 0 | 0 | 0 | 0 | 0 | 0 | 0 |
| NA13 | 0 | 0 | 0 | 0 | 0 | 0 | 0 |
| TESTIS | 100% | 100% | 100% | 100% | 100% | 100% | 100% |
| MB49 | 70 | 47 | 79 | 7 | 0.06 | 0.06 | 0.63 |
| MB-C2 | 0 | 0 | 0 | 0 | 0 | 0 | 0 |
| MB-C3 | 0 | 0 | 0 | 0 | 0 | 0 | 0 |
| MB-C5 | 0 | 0 | 0 | 0 | 0 | 0 | 0 |
| MB-C6 | 0 | 0 | 0 | 0 | 0 | 0 | 0 |
| MB-C9 | 0 | 0 | 0 | 0 | 0 | 0 | 0 |
| MB-C10 | 0 | 0 | 0 | 0 | 0 | 0 | 0 |
| MB-C12 | 0 | 0 | 0 | 0 | 0 | 0 | 0 |
| MB-C14 | 0 | 0 | 0 | 0 | 0 | 0 | 0 |
| MB-C18 | 0 | 0 | 0 | 0 | 0 | 0 | 0 |
| MB-C21 | 0 | 0 | 0 | 0 | 0 | 0 | 0 |
| MB-C22 | 0 | 0 | 0 | 0 | 0 | 0 | 0 |
| MB-C24 | 0 | 0 | 0 | 0 | 0 | 0 | 0 |
| MBC27 | 0 | 0 | 0 | 0 | 0 | 0 | 0 |
| MB-C32 | 0 | 0 | 0 | 0 | 0 | 0 | 0 |
| MB-C33 | 0 | 0 | 0 | 0 | 0 | 0 | 0 |
| MB-C36 | 0 | 0 | 0 | 0 | 0 | 0 | 0 |
| MB-C1 | 4 | 5 | 10 | 2 | 0 | 0.04 | 0 |
| MB-C29 | 5 | 10 | 15 | 5 | 0.00 | 0 | 0.18 |
| MB-C7 | 6 | 4 | 9 | 3 | 0.01 | 0 | 0.06 |
| MB-C20 | 17 | 10 | 26 | 8 | 0.00 | 0.03 | 0.29 |
| MB-C31 | 14 | 11 | 22 | 8 | 0.01 | 0.04 | 0.34 |
| MB-C16 | 16 | 16 | 23 | 8 | 0.00 | 0.02 | 0.2 |
| MB-C25 | 23 | 17 | 25 | 14 | 0.00 | 0.04 | 0.27 |
| MB-C13 | 50 | 35 | 80 | 29 | 0.00 | 0.06 | 0.95 |
| MB-C4 | 70 | 46 | 81 | 30 | 0.02 | 0.09 | 1.01 |
| MB-C8 | 123 | 74 | 156 | 48 | 0.02 | 0.12 | 1.82 |
| MB-C28 | 107 | 93 | 151 | 37 | 0.04 | 0.07 | 1.33 |
| MB-C17 | 10 | 118 | 243 | 62 | 0.02 | 0.15 | 1.85 |
| MB-C19 | 162 | 132 | 247 | 118 | 0.02 | 0.13 | 1.33 |
| MB-C30 | 321 | 109 | 253 | 60 | 0.02 | 0.02 | 0.94 |
| MB-C11 | 474 | 175 | 298 | 77 | 0.02 | 0.16 | 3 |
| MB-C34 | 529 | 179 | 193 | 153 | 0.03 | 0.05 | 0.97 |

### Impact of LOY on In Vitro and In Vivo BC Cell Growth

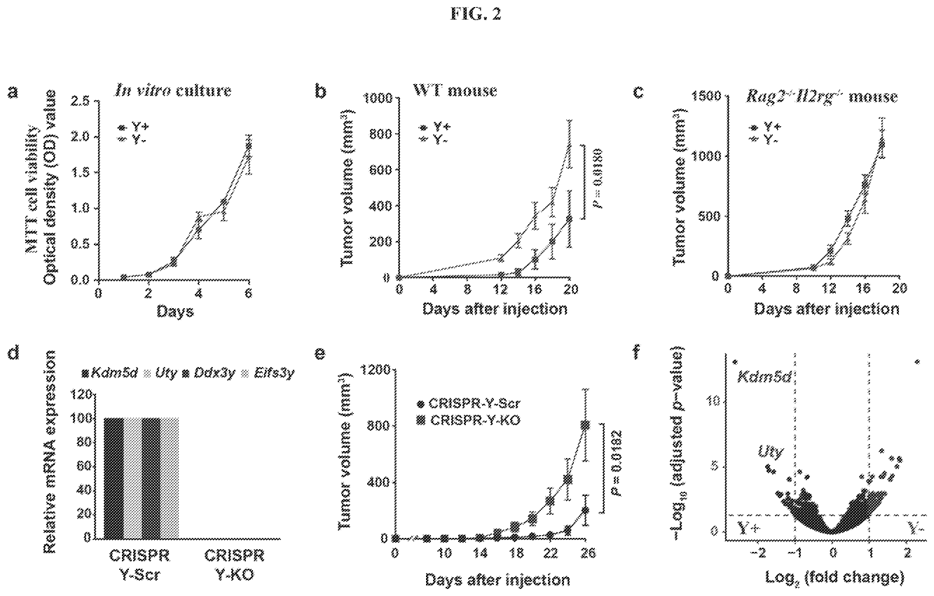

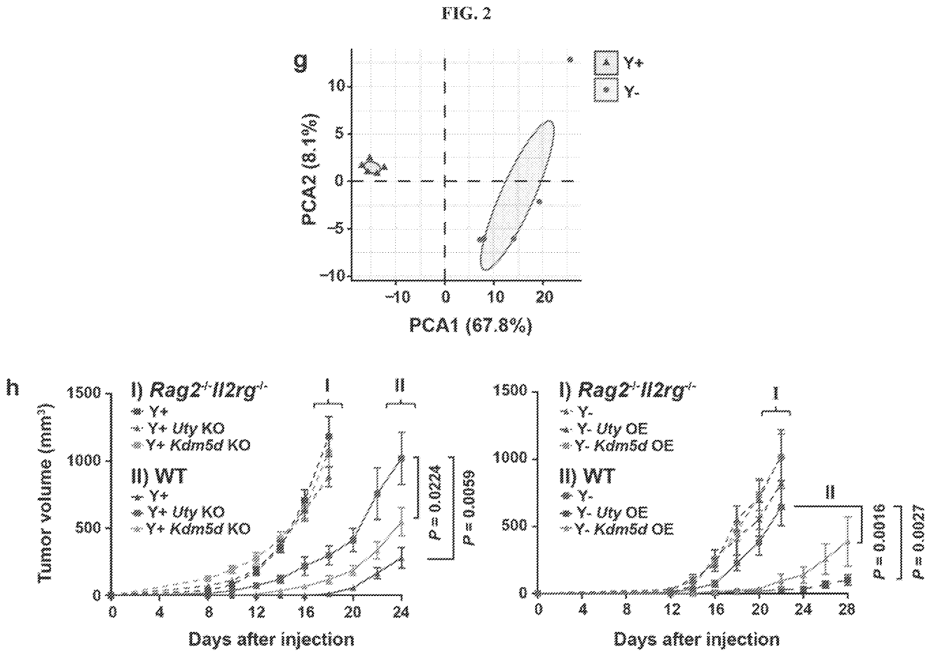

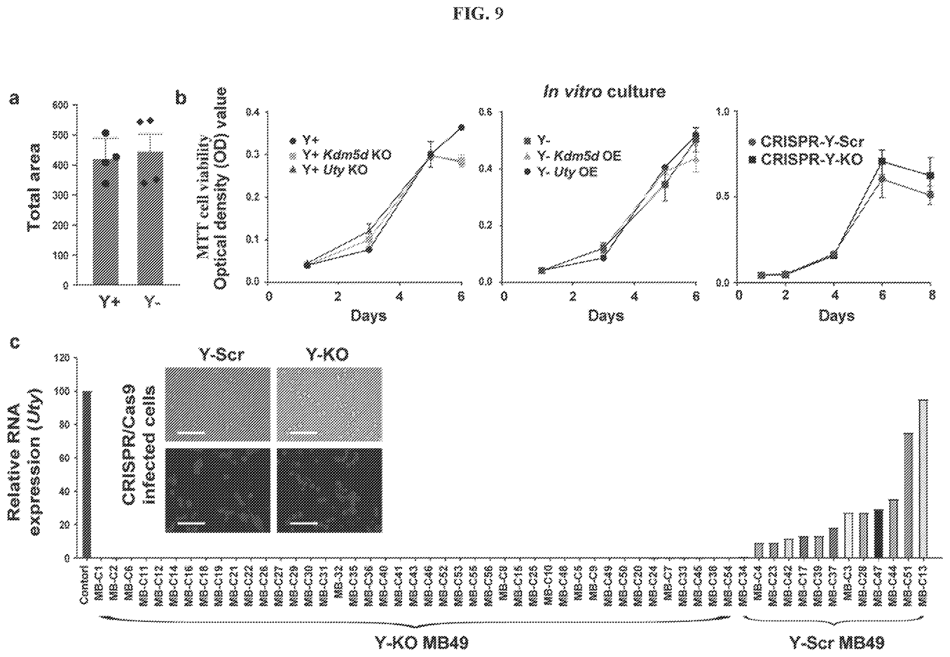

We observed no differences in proliferation between the Y− and Y+ sublines in either 2D or 3D in vitro culture (FIG. 2a and FIG. 9a-b). To investigate the impact of LOY on tumor growth in vivo, the Y− and Y+ lines were subcutaneously injected into immune competent wild type (WT) male C57BL/6 mice. We found that Y− tumors exhibited a ~2-fold increased tumor growth rate compared to Y+ tumors (FIG. 2b). This observation provided compelling clinical relevance for our model system, since it paralleled the relationship observed in human tumors described above. We then assessed the contribution of the host immunity to the differential growth by injecting each cell type into either WT or Rag2^{-/-}Il2rg^{-/-} (deficient in T cells, B cells, and NK cells) male mice. Again, Y− tumors grew significantly faster than Y+ tumors in the WT mice, whereas both cell types grew at the same rate in the immunocompromised Rag2^{-/-}Il2rg^{-/-} mice (FIG. 2c), indicating the Y− tumors were more efficient in evading anti-tumor adaptive immunity.

To separate Y chromosome loss from other potential differences in genetic background between the Y+ and Y− lines, we deleted the Y chromosome from Y+ cells using CRISPR/Cas9-mediated chromosome depletion, using previously established techniques (FIG. 2d). Of significance, we found that depletion of the Y chromosome (CRISPR-Y-KO) alone promoted faster tumor growth compared to the scrambled guide RNA Y+ control (CRISPR-Y-Scr) MB49 in immune competent mice (FIG. 2e and FIG. 9b-c). This essentially reproduced the findings of naturally occurring Y− bladder cancer cells.

We next aimed to define the molecular drivers in Y− tumors that contribute to immune evasion. Of the three genes shared between humans and MB49, low expression of KDM5D and UTY were the only human genes whose expression that when lost was associated with an unfavorable prognosis in human BC (FIG. 2f-g, FIG. 7c and Table 1). Therefore, we engineered Y+ and Y− sublines where Uty or Kdm5d was either knocked out or overexpressed. We found that Uty or Kdm5d single KO Y+ tumors exhibited an increased growth rate compared to Y+ tumors—a difference that was not observed in Rag2^{-/-}Il2rg^{-/-} mice (FIG. 2h). Consistent with these results, OE of either Uty or Kdm5d in Y− tumors resulted in decreased tumor growth in the WT but not in Rag2^{-/-}Il2rg^{-/-} mice (FIG. 2h). Again, all lines shared the same growth kinetics in vitro (FIG. 9b). Together, these results demonstrate that Uty and Kdm5d contribute, at least in part, to the impaired anti-tumor immunity of Y− tumors. Further, identification of KDM5D or UTY loss in human tumors has the potential to be a useful prognostic factor in determining the clinical aggressiveness of BC.

### LOY Cancer Cells Create an Immunosuppressive TME

To further determine the immunological basis of differential tumor growth of Y− vs. Y+ tumors, we sequenced the entire transcriptome from Y− and Y+ MB49 grown in WT mice. Nine hundred and fifty-eight differentially expressed genes were noted (FIG. 10a). Tumors from each genotype were separated on principal component analysis (PCA) (FIG. 10b), supporting a different molecular architecture. Gene set enrichment analyses (GSEA) performed on differentially expressed genes revealed Y+ tumors associated significantly with enhanced immune responses compared to Y− tumors (Positive regulation of Lymphocyte Proliferation: normalized enrichment score (NES)=1.59, Adjusted P-value=0.01; Lymphocyte Mediated Immunity: NES=1.76, Adjusted P-value=0.01; Response to Interferon-gamma: NES=1.87, Adjusted P-value=0.01; Positive Regulation of T Cell Activation: NES=1.80, Adjusted P-value=0.01) (FIG. 10c). We then further genetically defined the roles of T cells and B cells using mice with deficiency in each cellular compartment. We observed similar enhanced growth of Y− tumors in WT as well as in B cell deficient Ighm KO mice (FIG. 3a). However, differential tumor growth between Y+ and Y− tumors was eliminated in Rag2 and Tcrb/Tcrd KO mice, confirming that the increased tumor growth control of Y+ tumors was due to endogenous anti-tumor T cell immunity (FIG. 3a).

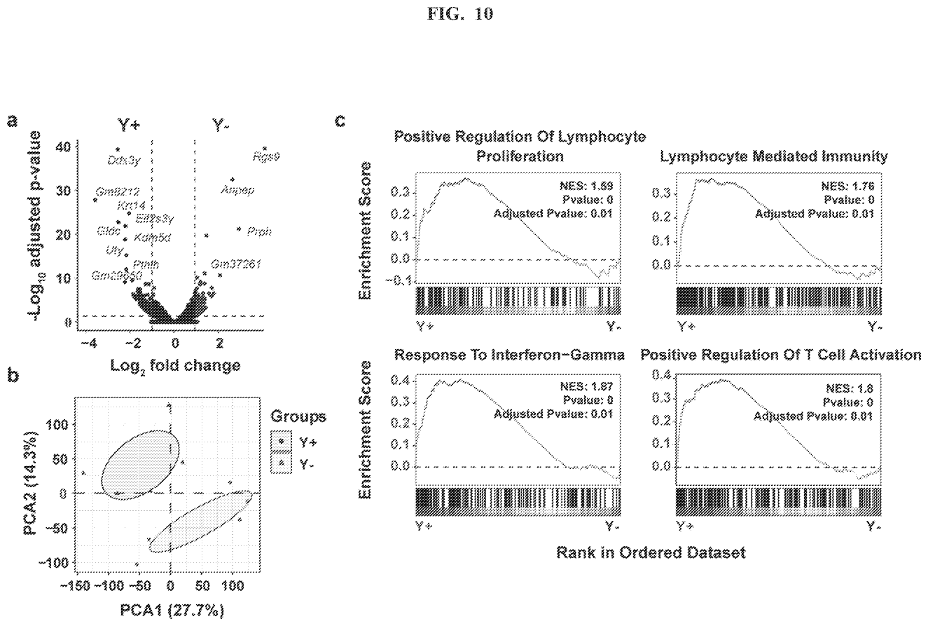

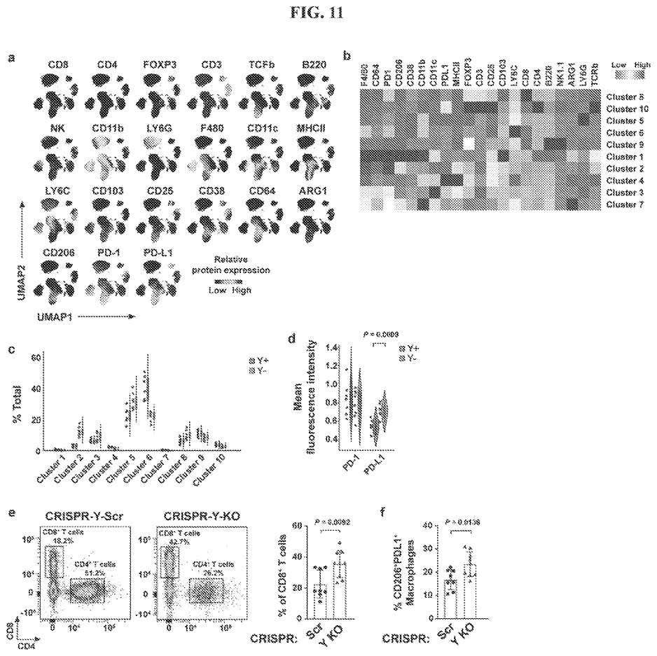

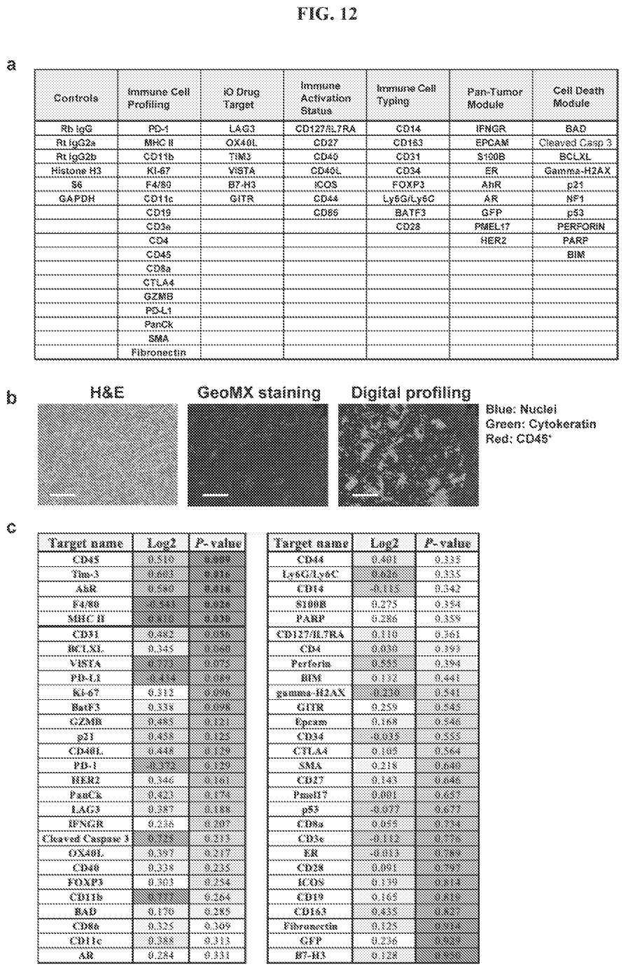

We next used high-dimensional spectral flow cytometry to delineate the difference in intratumoral CD45⁺ immune cell populations between Y+ and Y− tumors. We found that Y− tumors were enriched in the proportion of total CD8⁺ T cells (FIG. 3b-e and FIG. 11a-c) as well as immunosuppressive (CD11b⁺F4/80⁺LY6C⁻CD206⁺) macrophages. In comparison, tumor-infiltrating macrophages in Y+ tumors had a more inflammatory (CD11b⁺F4/80⁺LY6C⁺CD206⁻) phenotype (FIG. 3b-e and FIG. 11a-c). Of note, the percentage of PD-L1 positive CD45⁺ immune cells (FIG. 3f), as well as PD-L1 expression levels (FIG. 11d), was elevated in Y− tumors. Similar results were found in CRISPR-Y-Scr and Y-KO MB49 tumors in that the Y KO tumors contained a greater proportion of CD8⁺ T cells as well as immunosuppressive CD206⁺PDL1⁺ macrophages (FIG. 11e-f). Consistent with these findings, elevated F4/80⁺ macrophages and increased PD1/PD-L1 expression were also observed histologically in Y− MB49 tissue sections through GeoMX hi-plex spatial proteomic analysis (FIG. 3g and FIG. 12a-c).

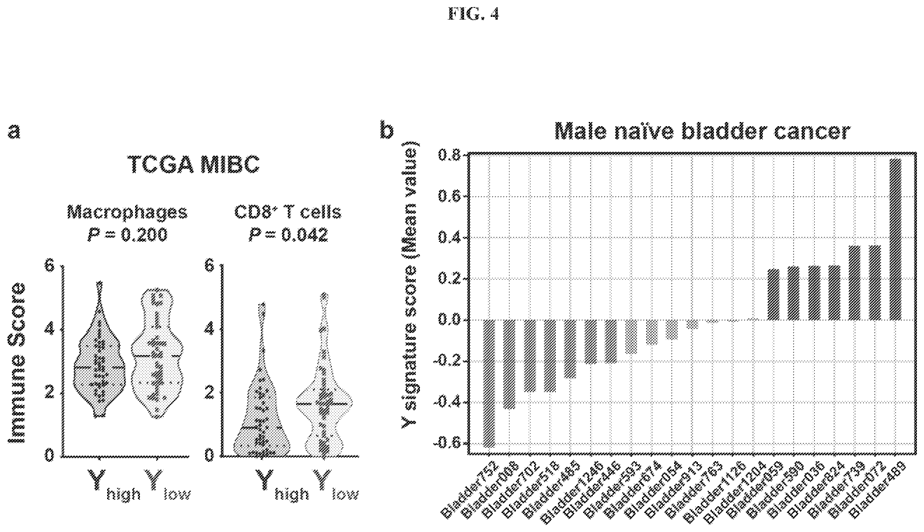

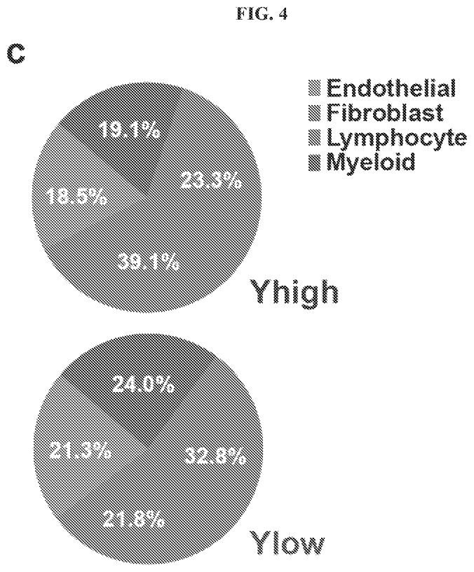

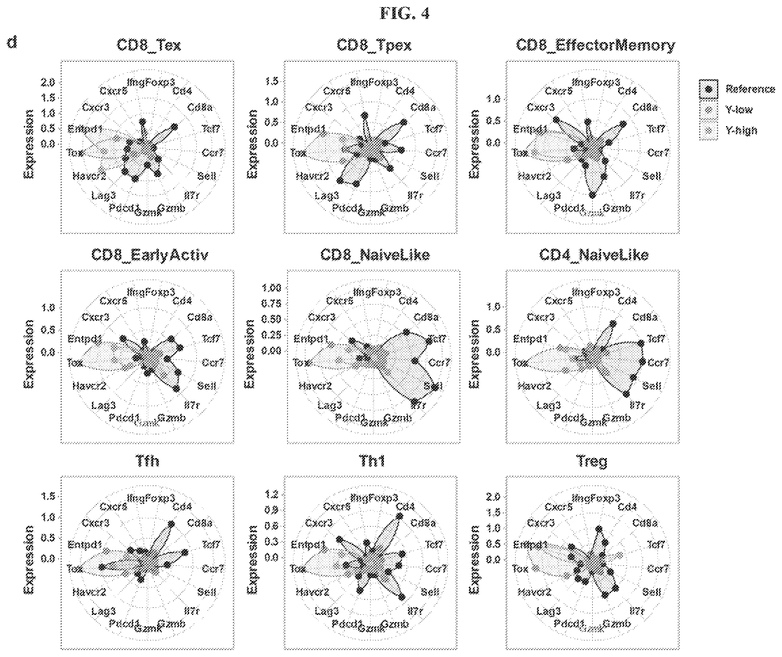

To determine if the TME of Y− murine tumors reflect the clinical picture, we deconvoluted urothelial BC RNA-seq from the TCGA database and mined a previously published MIBC snSeq dataset. Similar to our Y− vs. Y+ MB49 analyses, CD8⁺ T cells in Y_{low} tumors had increased expression of immune checkpoint molecules, such as CD274 (encoding PD-L1), LAG3, and HAVCR2 (encoding TIM3), as well as markers for both progenitor (TCF7) and terminal exhausted T cells (TOX) (FIG. 3h). We then compared proportions of these T cell subsets between tumor types by overlaying each cell sequenced with an annotated reference UMAP. Y_{low} BCs contained a higher proportion of exhausted and progenitor exhausted CD8⁺ T cells, as well as T regulatory cells (Tregs) (FIG. 3i). To further validate the snSeq results and to assess levels of T cell exhaustion histologically, we evaluated immune-focused CO-Detection by indEXing (CODEX) results from patient-matched samples. Consistent with our spectral flow cytometry and snSeq analyses, Y_{low} BCs contained a higher proportion of dysfunctional CD8⁺ T cells (FIG. 3j). Of significance, not only were CD8⁺ T cells present at higher proportions in Y_{low} vs. Y_{high} BC samples (FIG. 4a-c), but TOX expression was found to be elevated in all intratumoral T cell subsets of Y_{low} tumors, including CD8⁺ T cells (naïve-like, early activated, exhausted, progenitor exhausted, and effector memory) and CD4+ T cells [Tregs, naïve-like, Tfh, and Th1cells](FIG. 4d). Altogether, the data support the notion of LOYalT, i.e., LOY tumors promote CD8⁺ T cell exhaustion in the TME.

### LOYBC Responds Better to Anti-PD-1/PD-L1 ICB Therapy

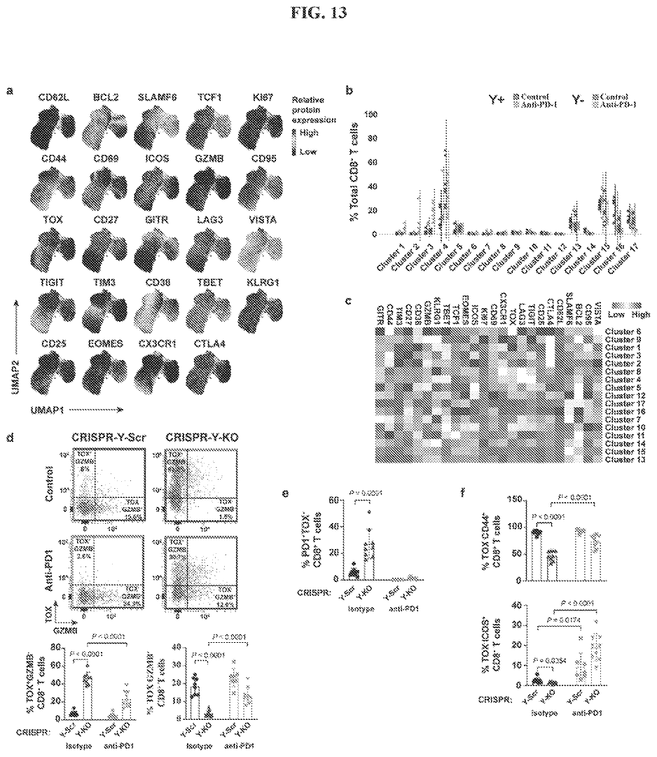

Given that an increase in exhausted and progenitor exhausted CD8⁺ T cells in the TME is associated with superior response to anti-PD-1/PD-L1 ICB, we treated the Y+ and Y− MB49 lines, as well as the CRISPR-Y-Scr and Y-KO MB49 lines, with anti-PD-1 blocking antibodies. At the end of the treatment course, we performed spectral flow cytometry to examine the dynamics of tumor-infiltrating CD8⁺ T cells with and without ICB. Both Y− tumor models (i.e., clonally selected and CRISPR generated) demonstrated a superior response to anti-PD1 treatment compared to Y+ tumors (FIG. 5a). Consistent with our human snSeq analyses (FIG. 3h-i), CD8⁺ T cells from isotype control antibody-treated Y− tumors had elevated expression of exhaustion markers, such as TOX and TIM3, compared to Y+ tumors (FIG. 5b-d and FIG. 13a). Importantly, CD8⁺ T cells from Y− tumors demonstrated a greater phenotypic response to anti-PD1 treatment (FIG. 5d and FIG. 13b)—from a more exhausted (expressing TIM3, LAG3 and TOX) to a more activated (expressing CD44, ICOS but not TOX) differentiation state (FIGS. 5a and 5b and FIG. 13d-f). The less aggressive nature of Y+ tumors can be best seen at earlier stages of tumor growth (FIGS. 2b and 2e), which we attribute to a baseline more activated lymphocytic cell state at baseline. This can be seen at both the RNA (FIG. 11) and protein level (FIGS. 13d and 13f) and is likely the reason these tumors do not respond as well to anti-PD-1 treatment.

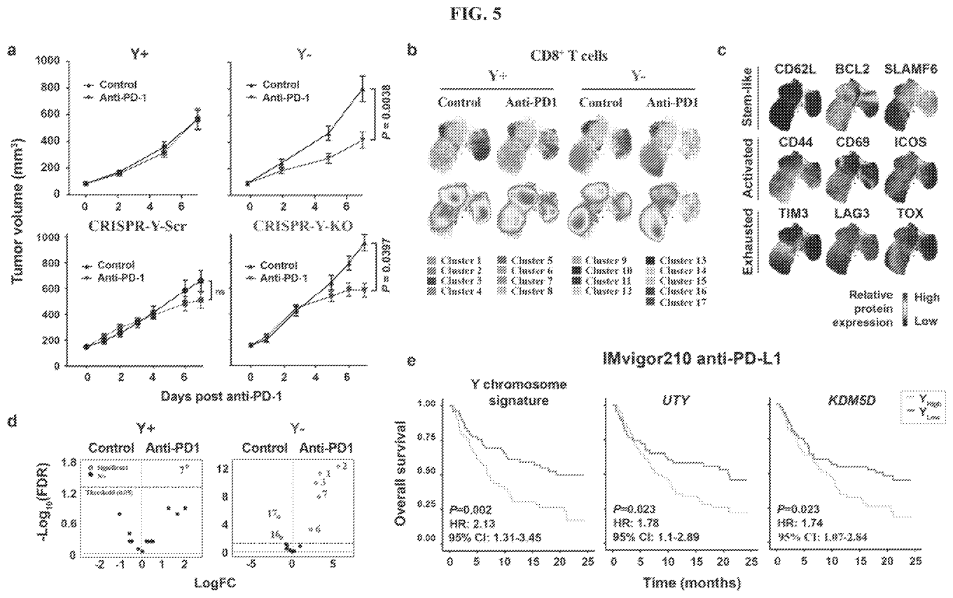

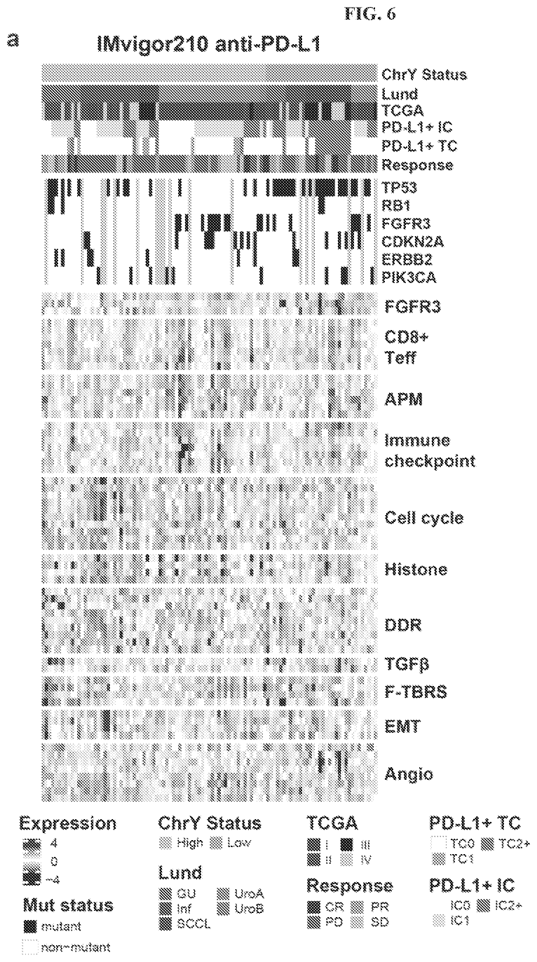

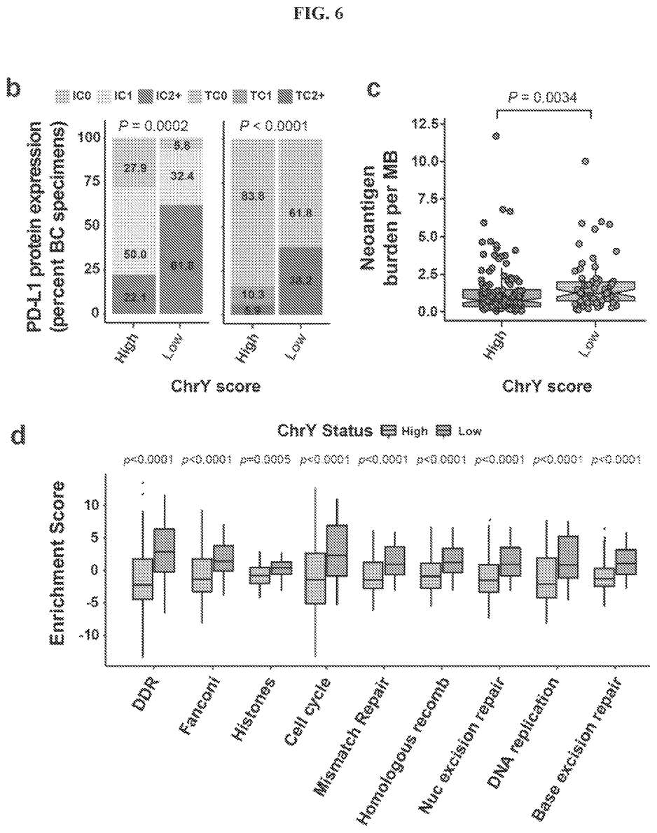

To determine the human relevance of our preclinical work, we evaluated overall survival of Y_{low} versus Y_{high} BC patients from the IMvigor210 atezolizumab (anti-PD-L1) clinical trial. Consistent with a superior response of Y− MB49 tumors to anti-PD-1 treatment, Y_{low} BC patients had better outcomes after anti-PD-L1 treatment (FIG. 5e). Compellingly, similar outcomes were observed if only stratifying patients based on UTY or KDM5D expression, underscoring the important role a loss of these two genes has in conferring the LOY phenotype (FIG. 5e). Similar to observations made in our murine models (see FIG. 3f and FIG. 11d) and human BC specimen evaluations (see FIG. 3h), PD-L1 levels were also enriched in Y_{low} BC from the IMvigor210 trial (FIG. 6a-b).

Of relevance, a recent report demonstrated that LOY and the loss of KDM5D induced increased genomic instability in hematopoietic stem and progenitor cells (HSPCs). Therefore, we evaluated DNA replication and repair pathway activation between tumor types. Pathways including DNA damage repair (DDR), mismatch repair, and nucleotide excision repair were all elevated in Y_{low} tumors (FIG. 6c-d), indicating that these tumors were indeed more genomically unstable. Further genetic evaluations of Y_{low} and Y_{high} BC from this cohort revealed an increase in tumor neoantigen burden (TNB) in Y_{low} tumors that likely contributed to their increased response to ICB (FIGS. 6a and 6c).

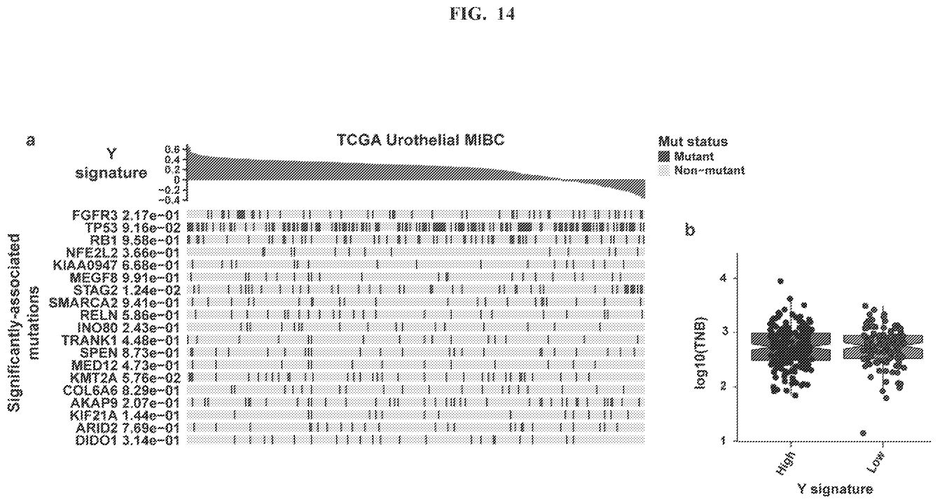

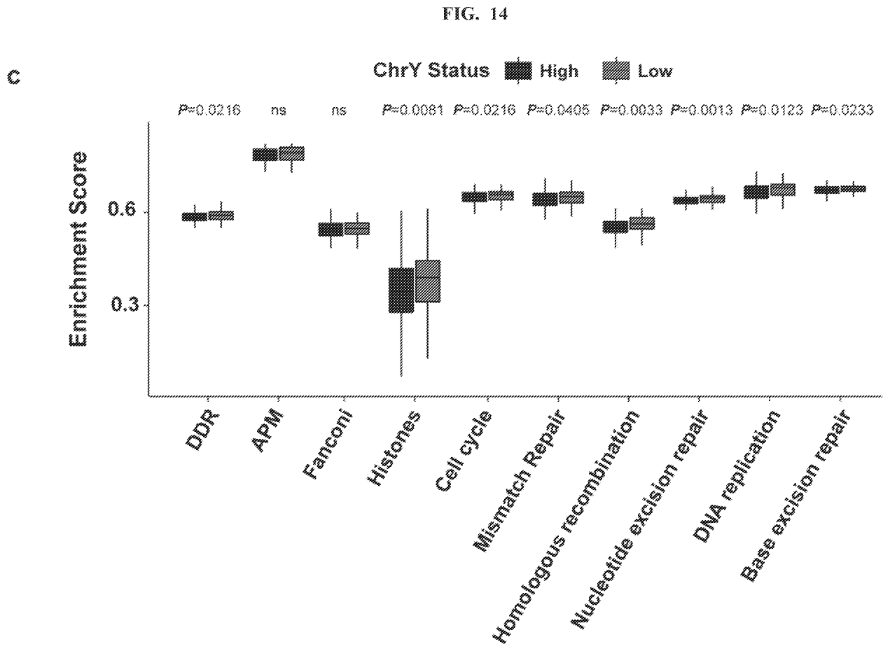

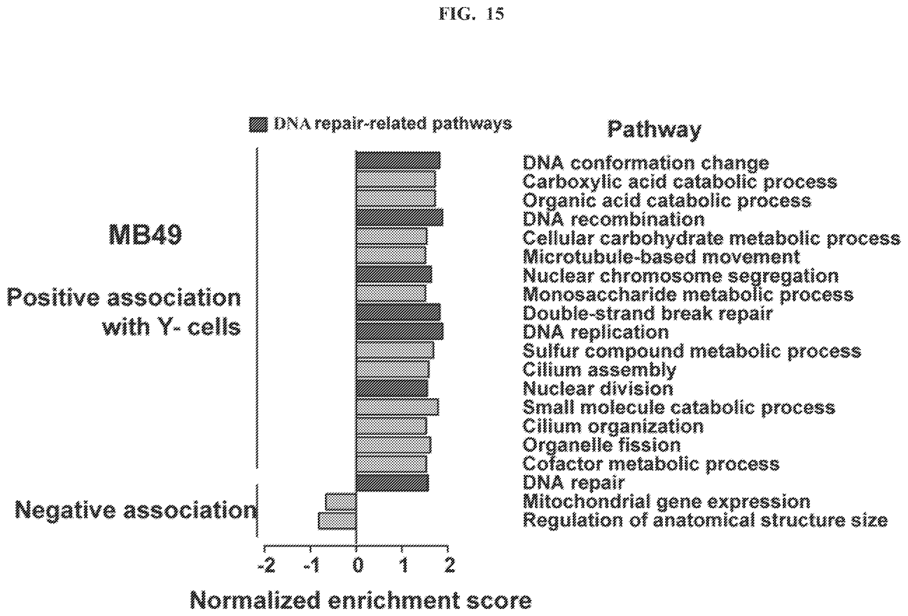

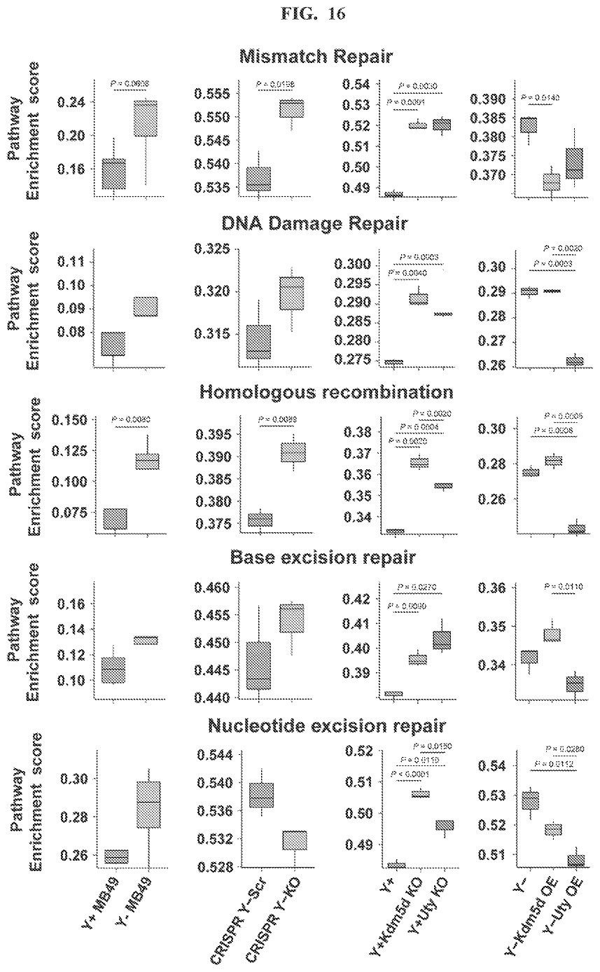

Aside from an increase in TNB, increased DDR pathway activation was also present in the Y_{low} TCGA tumors (FIG. 14a-c) and in our Y− MB49 cell line (FIGS. 15 and 16). Collectively, this is the first evidence demonstrating LOY in bladder tumor cells contributes to the aggressive nature of Y_{low} BC, which can be attributed to effective evasion of T cell immunity secondary to an increased presence of PD-L1-expressing macrophages and ensuing CD8⁺ T cell exhaustion. Importantly, consistent with the LOYalT phenomenon we uncovered in this study, Y− tumors were also found for the first time to be more responsive to anti-PD-1 immune checkpoint blockers (ICBs), underscoring the translational significance of our findings.

### Example 3

A database of patients which have been treated with PD-1/PD-L1 inhibitors was queried and identified 171 males who have these therapies (Atezolizumab (PD-L1)-16, Avelumab (PD-L1)-41, Durvalumab (PD-L1)-4, Nivolumab (PD-1)-1, Pembrolizumab (PD-1)-109) for advanced or metastatic bladder cancer either as first line or second line therapy.

171 bladder cancer patients, Y-high group and LOY group, were all treated with PD-1/PD-L1 inhibitors. The PD-i/PD-L1 inhibitors that these patients were treated with included: atezolizumab: 16; avelumab: 41; durvalumab: 4; nivolumab: 1; pembrolizumab: 109. Time is calculated in days—from the regimen start date to the last known date of follow up. The outcome showed 77 dead and 94 alive.

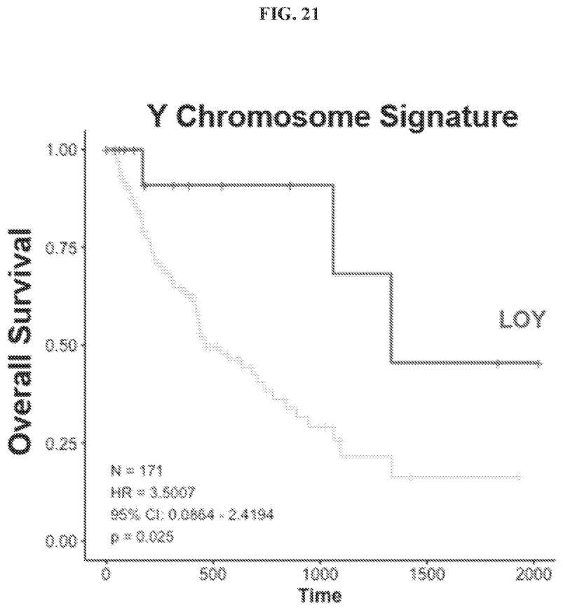

As seen in FIG. 21, the LOY group of patients is more responsive, and thus, have higher survival.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of selecting a cancer treatment for a male subject, comprising:
assaying a biological sample obtained from the male subject to determine a Y chromosome gene expression signature;
detecting a loss of Y chromosome (LOY) phenotype, and selecting a therapy comprising an immune checkpoint inhibitor, a TOX inhibitor, or a combination thereof, or
detecting a Y chromosome high phenotype, and selecting a therapy comprising an immune checkpoint inhibitor and an inhibitor of one or more genes on the Y Chromosome.

2. The method of claim 1, wherein the cancer is bladder cancer (BLCA), colon adenocarcinoma microsatellite instability (COAD MSI), skin cutaneous melanoma (SKCM), kidney renal clear cell carcinoma (KIRC), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD).

3. The method of claim 1, wherein detecting LOY phenotype comprises detecting a low level of one or more genes on the Y chromosome, as compared to each gene's reference level.

4. The method of claim 3,
wherein the one or more genes comprise BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY, or
wherein the one or more genes comprise BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

5. (canceled)

6. The method of claim 3,
wherein the one or more genes comprise KDM5D, UTY (KDM6C), TBL1Y, ZFY, or a combination thereof, or
wherein the one or more genes comprise UTY, KDM5D, or both, or
wherein the one or more genes is NLGN4Y.

7. (canceled)

8. (canceled)

9. The method of claim 1, wherein the biological sample is a tumor sample.

10. The method of claim 1, wherein the immune checkpoint inhibitor is an anti-PD-L1 inhibitor or an anti-PD-1 inhibitor.

11. The method of claim 1, wherein the immune checkpoint inhibitor is atezolizumab.

12. The method claim 1, further comprising administering the selected therapy.

13. A method of treating cancer in a male subject, comprising:
administering a therapy comprising an immune checkpoint inhibitor, a TOX inhibitor, or a combination thereof to the male subject, wherein the male subject has been detected to have a loss of Y chromosome (LOY) phenotype,
or
administering a therapy comprising immune checkpoint inhibitor and an inhibitor of one or more genes on the Y chromosome to the male subject, wherein the male subject has been detected to have a Y chromosome high phenotype.

14. The method of claim 13, wherein the cancer is bladder cancer (BLCA), colon adenocarcinoma microsatellite instability (COAD MSI), skin cutaneous melanoma (SKCM), kidney renal clear cell carcinoma (KIRC), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD).

15. The method of claim 13,
wherein the inhibitor is an inhibitor of BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY, or
wherein the inhibitor is an inhibitor of BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

16. (canceled)

17. The method of claim 13,
wherein the inhibitor is an inhibitor of KDM5D, UTY (KDM6C), TBL1Y, or ZFY, or a combination thereof, or
wherein the inhibitor is an inhibitor of UTY, KDM5D, or both, or
wherein the inhibitor is an inhibitor of NLGN4Y.

18. (canceled)

19. (canceled)

20. The method of claim 13, wherein the immune checkpoint inhibitor is an anti-PD-L1 inhibitor or an anti-PD-1 inhibitor.

21. The method of claim 13, wherein the immune checkpoint inhibitor is atezolizumab.

22. A method of treating cancer in a male subject, comprising:
requesting the results of an assay of a biological sample obtained from the male subject to determine a Y chromosome gene expression signature;
administering a therapy comprising an immune checkpoint inhibitor, a TOX inhibitor, or a combination thereof to the male subject, wherein the male subject has been detected to have a loss of Y chromosome (LOY) phenotype,
or
administering a therapy comprising immune checkpoint inhibitor and an inhibitor of one or more genes on the Y chromosome to the male subject, wherein the male subject has been detected to have a Y chromosome high phenotype.

23. The method of claim 22, wherein the cancer is bladder cancer (BLCA), colon adenocarcinoma microsatellite instability (COAD MSI), skin cutaneous melanoma (SKCM), kidney renal clear cell carcinoma (KIRC), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD).

24. The method of claim 22, wherein the LOY phenotype was detected by detecting a low level of one or more genes on the Y chromosome, as compared to each gene's reference level.

25. The method of claim 22,
wherein the one or more genes comprise BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY, or
wherein the one or more genes comprise BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

26. (canceled)

27. The method of claim 22, wherein the one or more genes comprise KDM5D, UTY (KDM6C), TBL1Y, ZFY, or a combination thereof, or
wherein the one or more genes comprise UTY, KDM5D, or both, or
wherein the one or more genes is NLGN4Y.

28. (canceled)

29. (canceled)

30. The method of claim 22, wherein the biological sample is a tumor sample.

31. The method of claim 22,
wherein the inhibitor is an inhibitor of BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY or
wherein the inhibitor is an inhibitor of BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A.

32. (canceled)

33. The method of claim 22,
wherein the inhibitor is an inhibitor of KDM5D, UTY (KDM6C), TBL1Y, ZFY, or a combination thereof, or
wherein the inhibitor is an inhibitor of UTY, KDM5D, or both.

34. (canceled)

35. The method of claim 22, wherein the immune checkpoint inhibitor is an anti-PD-L1 inhibitor or an anti-PD-1 inhibitor.

36. The method of claim 22, wherein the immune checkpoint inhibitor is atezolizumab.

37. A method of determining susceptibility to treatment with an immune checkpoint inhibitor, a TOX inhibitor, or both, in a male subject having cancer, comprising
assaying a biological sample obtained from the male subject to determine a Y chromosome gene expression signature;
detecting a loss of Y chromosome (LOY) phenotype, wherein the LOY indicates a susceptibility treatment with an immune checkpoint inhibitor, a TOX inhibitor, or both, or
detecting a Y chromosome high phenotype, wherein the Y chromosome high phenotype indicates a low likelihood of susceptibility treatment with an immune checkpoint inhibitor, a TOX inhibitor, or both, or indicates a benefit from a therapy comprising an immune checkpoint inhibitor and an inhibitor of the one or more genes on the Y chromosome,
wherein the biological sample is a tumor sample,
wherein the cancer is bladder cancer (BLCA), colon adenocarcinoma microsatellite instability (COAD MSI), skin cutaneous melanoma (SKCM), kidney renal clear cell carcinoma (KIRC), liver hepatocellular carcinoma (LIHC), head and neck squamous cell cancer (HNSC), lung squamous cell carcinoma (LUSC), lung adenocarcinoma (LUAD), mesothelioma (MESO), esophageal carcinoma (ESCA), sarcoma (SARC), glioblastoma multiforme (GBM), or prostate adenocarcinoma (PRAD),
wherein the one or more genes comprise BPY1, BPY1B, BPY2, BPY2B, BPY2C, CDY1A, CDY1B, CDY2A, CSPG4LY, CYorf15A, CYorf15B, DAZ1, DAZ2, DAZ3, DAZ4, DDX3Y, EIF1AY, GOLGA1, GOLGA2LY, GOLGA3, GOLGA5, GOLGA6B, GOLGA6C, GOLGA6D, GOLGA6L7P, GOLGA7, HSFY1, HSFY2, KDM5D, NLGN4Y, PRY, PRY2, RBMY1A1, RPS4Y2, TMSB4Y, USP9Y, UTY, or XKRY, or wherein the one or more genes comprise BPY2, CDY1B, CYorf15A, CYorf15B, DAZ1, DAZ2, DAZ4, DAZ3, DDX3Y, EIF1AY, GOLGA1, GOLGA3, GOLGA5, GOLGA7, GOLGA6B, GOLGA6D, GOLGA6C, HSFY2, NLGN4Y, PRY2, RBMY1A1, RPS4Y2, KDM5D, TMSB4Y, USP9Y, UTY, XKRY, BPY1, or CDY1A, or
wherein the one or more genes comprise KDM5D, UTY (KDM6C), TBL1Y, ZFY, or a combination thereof, or
wherein the one or more genes comprise UTY, KDM5D, or both, or
wherein the one or more genes is NLGN4Y.

38. (canceled)

39. The method of claim 37, wherein detecting the LOY phenotype comprises detecting a low level of one or more genes on the Y chromosome, as compared to each gene's reference level.

40. (canceled)

41. (canceled)

42. (canceled)

43. (canceled)

44. (canceled)

45. (canceled)

46. The method of claim 37, wherein the immune checkpoint inhibitor is an anti-PD-L1 inhibitor or an anti-PD-1 inhibitor.

47. (canceled)
